(19) United States
(12) Patent Application Publication
(10) Pub. No.: US 2023/0416342 A1
(43) Pub. Date: Dec. 28, 2023

(54) METHODS FOR PURIFYING INTER-ALPHA INHIBITOR PROTEINS

(71) Applicant: ProThera Biologics, Inc., Providence, RI (US)

(72) Inventors: Yow-Pin LIM, Providence, RI (US); Joseph QIU, Providence, RI (US)

(21) Appl. No.: 18/036,720

(22) PCT Filed: Nov. 16, 2021
(86) PCT No.: PCT/US2021/059569

§ 371 Date: May 12, 2023

Related U.S. Application Data
(60) Provisional application No. 63/114,416, filed on Nov. 16, 2020.

(51) Int. Cl. C07K 14/81

(52) U.S. Cl. CPC C07K 14/811

(57) ABSTRACT

Described herein are methods for purifying IαIp from a biological material using an endotoxin-binding agent. The method involves applying the biological material containing the IαIp to an endotoxin-binding agent, discarding the flow through, applying a wash buffer(s), and eluting the IαIp from the endotoxin-binding agent.

### BACKGROUND

Inter-alpha Inhibitor Proteins (IαIps) are a family of naturally occurring, immunomodulatory plasma proteins that circulate in high concentrations in the blood of all mammals. IaαIps promote protective effects against inflammation caused by infection, trauma, and injury. The protective effects of IaαIps are independent from the causative microbial agents or triggers.

Members of this family are composed of heavy and light polypeptide subunits that are covalently linked by glycosaminoglycan. IaαIps can be found in vivo as Inter-alpha-Inhibitor (IαI), a 250 kDa molecule composed of two heavy chains (H1 & H2) and a single light chain (L), termed bikunin, and Pre-alpha-Inhibitor (PαI), a 125 kDa molecule composed of one heavy (H3) and one light chain (L).

When the body generates inflammatory signals, such as those elicited during injury or infection, IaαIps traffic into the tissues and directly reach sites of inflammation. The heavy chains of IaαIps enhance the anti-inflammatory response by binding to proteins which are part of the inflammatory cascade, such as complement and extracellular histones (Damage Signals), thereby attenuating inflammatory processes, while the light chain bikunin inhibits the activity of serine proteases, such as trypsin, elastase, plasmin, cathepsin G, and furin.

IaαIps have been shown to promote lung epithelial repair after injury in both in vitro and in vivo models and IaαIps have also been shown in multiple in vivo models to down regulate inflammatory cytokines, such as TNF-α and IL-6.

In healthy individuals, the amount of circulating IαIp in blood is relatively high (between 400-800 mg/L). IαIp levels rapidly decrease during systemic inflammation/sepsis in newborns and in adult patients (Baek Y W, et al. J Pediatr. 2003; 143:11-15; Lim Y P, et al. J Infect Dis. 2003; 188:919-926 and Opal S M, et al. Crit Care Med. 2007; 35:387-392), and decreased levels of IαIp have been shown to correlate strongly with disease progression. IαIp therapy has been described for the treatment of sepsis and the associated organ damage, pneumonia, acute respiratory disease, necrotizing enterocolitis (NEC), wounds, burns, cancer, stroke, and Alzheimer's disease.

Previously, IaαIps were prepared using stepwise extraction followed by chromatographic separations. While these methods can achieve high purity (e.g., >90% purity), the methods suffer from low yield of IaαIps (e.g., 20-30% (w/w)). Thus, there exists a need for methods for purifying or preparing IaαIps in high yield and purity for use, for example, in the preparation of therapeutic compositions.

### SUMMARY OF THE DISCLOSURE

The disclosure features methods of purifying an IαIp (e.g., one or more of IαI, PαI, and bikunin). The methods involve applying a biological material containing an IαIp (e.g., blood or milk), such as a biological material obtained from a subject (e.g., a human), to an endotoxin-binding agent (e.g., a solid support, such as a chromatography column, containing an endotoxin-binding agent) as a step in a purification process.

A first aspect of the disclosures features a method of purifying an inter-alpha inhibitor protein (IαIp) from a biological material by: (a) applying the biological material comprising the IαIp to an endotoxin-binding agent and separating a flow through comprising the biological material that does not bind to the endotoxin-binding agent; and (b) applying an elution buffer comprising a salt to the endotoxin-binding agent and collecting an eluate comprising the IαIp.

In several embodiments, the endotoxin-binding agent is immobilized on a support (e.g., a monolithic support or a particle-based support). In several embodiments, the monolithic support or particle-based support is or comprises a resin. The support may be, for example, a column, membrane, disc, or chip.

In other embodiments, the endotoxin-binding agent is selected from the group consisting of ETOXICLEAR™, PIERCE™ High Capacity Endotoxin Removal Resin, TOXINERASER™ Endotoxin Removal Resin, PURKINE™ Endotoxin Removal Resin, DETOXI-GEL™ Endotoxin Removing Gel, and PROMEGA™ Endotoxin Removal Resin. In particular embodiments, the endotoxin-binding agent is DETOXI-GEL™ or ETOXICLEAR™.

The biological material may contain three or more proteins selected from the group consisting of alpha-1 antitrypsin, C1-inhibitor, albumin, a globulin (e.g., an immunoglobulin (e.g., IgA, IgE, IgM, IgD, and IgG (e.g., intravenous Ig (IVIg), anti-D IgG, hepatitis B IgG, measles IgG, rabies IgG, tetanus IgG, and Varicella Zoster IgG))), fibrinogen (factor I), prothrombin (factor II), thrombin, anti-thrombin III, factor III, factor V, factor VII, factor VIII, factor IX, factor X, factor XI, factor XII, factor XIII, fibronectin, alpha-2 antiplasmin, urokinase, protein C, protein S, protein Z, protein Z-related protease inhibitor, plasminogen, tissue plasminogen activator, plasminogen activator inhibitor-1, plasminogen activator inhibitor-2, von Willebrand factor, factor H, prekallikrein, high-molecular-weight kininogen, and heparin cofactor II.

In some embodiments, the biological material comprises three to ten, three to fifteen, three to twenty, three to twenty five, three to thirty, ten to twenty, ten to twenty five, ten to thirty, fifteen to twenty five, fifteen to thirty, twenty to thirty, or thirty or more different proteins.

In some embodiments, the biological material comprises about 40 to about 65% albumin (w/w) and/or about 25 to about 45% globulins (w/w) and/or about 2 to about 12% fibrinogen (w/w).

In some embodiments, the method further comprises applying a first wash buffer to the endotoxin-binding agent after step (a) and prior to step (b). In some embodiments, the method further comprises separating a flow through comprising the first wash buffer. In some embodiments, the first wash buffer has a pH of about 4.5 to 8.5 (e.g., about pH 5.2). In some embodiments, the first wash buffer comprises one or more of glycine, acetic acid, citric acid, phosphate, sodium chloride (NaCl), calcium, magnesium, EDTA, and Tris-HCl. In some embodiments, the first wash buffer comprises about 10 to about 200 mM glycine and/or about 20 to 300 mM acetic acid (e.g., 75 mM glycine and about 100 mM acetic acid). In some embodiments, the first wash buffer comprises about 200 mM or less NaCl (e.g., about 50 to about 150 mM NaCl (e.g., about 50 mM NaCl or about 100 mM NaCl)). In some embodiments, the first wash buffer comprises about 100 to about 300 mM NaCl (e.g., about 200 mM NaCl). In some embodiments, the first wash buffer comprises about 75 mM glycine, about 100 mM AcOH and about 200 mM NaCl. In some embodiments, the method further comprises applying a second wash buffer to the endotoxin-binding agent after applying the first wash buffer. In some embodiments, the method further comprises separating a flow through comprising the second wash buffer. In some embodiments, the second wash buffer has a pH of about 4.5 to about 8.5 (e.g., about pH 7.2). In some embodiments, the second wash buffer comprises one or more of glycine, acetic acid, citric acid, phosphate, sodium chloride (NaCl), calcium, magnesium, EDTA, and Tris-HCl. In some embodiments, the second wash buffer comprises about 5 to about 100 mM Tris-HCl. (e.g., about 20 mM Tris-HCl). In some embodiments, the second wash buffer comprises about 500 mM NaCl or less (e.g., about 100 to about 500 mM NaCl (e.g., about 300 mM NaCl)). In some embodiments, the second wash buffer comprises about 200 to about 400 mM NaCl (e.g., about 300 mM NaCl)). In some embodiments, the second wash buffer comprises about 20 mM Tris and about 300 mM NaCl.

In some embodiments, the method further comprises applying a third wash buffer to the endotoxin-binding agent after applying the second wash buffer. In some embodiments, the elution buffer has a pH of about 4.5 to about 8.5 (e.g., about pH 7.2). In some embodiments, the elution buffer comprises one or more of glycine, acetic acid, citric acid, phosphate, sodium chloride (NaCl), calcium, magnesium, EDTA, and Tris-HCl. In some embodiments, the elution buffer comprises about 5 to about 100 mM Tris-HCl (e.g., about 20 mM Tris-HCl). In some embodiments, the elution buffer comprises about 1,000 mM NaCl or less (e.g., about 500 to about 1,000 mM NaCl (e.g., about 500 mM NaCl or about 1,000 mM NaCl)). In some embodiments, the elution buffer comprises about 20 mM Tris and about 500 mM NaCl.

In some embodiments, the method further comprises applying a second elution buffer to the endotoxin-binding agent after applying the elution buffer, the second elution buffer has a pH of about 4.5 to about 8.5 (e.g., about pH 7.2). In some embodiments, the second elution buffer comprises one or more of glycine, acetic acid, citric acid, phosphate, sodium chloride (NaCl), calcium, magnesium, EDTA, and Tris-HCl. In some embodiments, the second elution buffer comprises about 5 to about 100 mM Tris-HCl (e.g., about 20 mM Tris-HCl). In some embodiments, the second elution buffer comprises about 1,000 mM NaCl or less (e.g., about 500 to about 1,000 mM NaCl (e.g., about 500 mM NaCl or about 1,000 mM NaCl)). In some embodiments, the second elution buffer comprises about 20 mM Tris and about 1,000 mM NaCl.

In some embodiments, the method further comprises applying a dilution buffer to the biological material prior to step (a). In some embodiments, the dilution buffer comprises deionized water. In some embodiments, the dilution buffer comprises purified water. In some embodiments, the dilution buffer has a pH of about 4.5 to about 8.5 (e.g., about pH 5.5, about pH 7.2, or about pH 7.3). In some embodiments, the biological material is diluted 1:1 to 1:10 (v/v) with the dilution buffer. In some embodiments, the dilution buffer comprises one or more of glycine, acetic acid, citric acid, phosphate, sodium chloride (NaCl), calcium, magnesium, EDTA, and Tris-HCl. In some embodiments, the dilution buffer comprises about 5 to about 100 mM Tris-HCl (e.g., about 20 mM Tris-HCl. In some embodiments, the dilution buffer comprises about 50 mM NaCl or less (e.g., no salt, no NaCl or about 50 mM NaCl). In some embodiments, the dilution buffer comprises about 5 to about 100 mM phosphate (e.g., 15 mM phosphate).

In some embodiments, the method further comprises adjusting pH of the biological material prior to step (a). In some embodiments, the method comprises adjusting pH of the biological material to about 4.5 to about 8.5 (e.g., about pH 5.5, about pH 7.2, or about pH 7.3) by addition of acetic acid prior to step (a). In some embodiments, the method further comprises adjusting conductivity of the biological material prior to step (a). In some embodiments, the method comprises adjusting conductivity of the biological material to about 10 to about 30 mS/cm, about 15 to about 25 mS/cm (e.g., about 20 mS/cm) prior to step (a).

In some embodiments, the method further comprises detecting an amount of the IαIp in the flow through. In some embodiments, the method comprises discarding the flow through.

In some embodiments, the method further comprises detecting an amount of IαIp in the eluate.

In some embodiments, the method comprises a flow rate of about 1 to 10 mL/minute.

In some embodiments, the method further comprises applying the biological material to a chromatography support (e.g., an anion-exchange chromatography support, a size-exclusion chromatography support, an ion-exchange chromatography support, an affinity chromatography support, or a combination thereof). In some embodiments, the chromatography support is said anion-exchange chromatography support.

In some embodiments, the method further comprises: (i) applying the biological material to the chromatography support and separating a flow through of step (i) comprising the biological material that does not bind to the chromatography support; and (ii) applying an elution buffer to the chromatography support and collecting an eluate comprising the IαIp.

In some embodiments, the chromatography support is a monolithic support or a particle-based support. In some embodiments, the monolithic support or particle-based support comprises an immobilized anion-exchange resin (e.g., diethylaminoethane (DEAE) resin or a quaternary amine (Q) resin). In some embodiments, the monolithic support or particle-based support comprises a quaternary amine (Q) resin. In some embodiments, the chromatography support is a column, membrane, disc, or chip.

In some embodiments, the method further comprises applying a first wash buffer to the chromatography support after step (i) and prior to step (ii). In some embodiments, prior to step (ii), the method further comprises separating a flow through comprising the first wash buffer. In some embodiments, the first wash buffer applied to the chromatography support has a pH of about 4.5 to about 8.5 (e.g., about 7.2). In some embodiments, the first wash buffer applied to the chromatography support comprises one or more of glycine, acetic acid, citric acid, phosphate, sodium chloride (NaCl), calcium, magnesium, EDTA, and Tris-HCl. In some embodiments, the first wash buffer applied to the chromatography support comprises about 5 to about 100 mM Tris-HCl (e.g., about 20 mM Tris-HCl). In some embodiments, the first wash buffer applied to the chromatography support comprises about 400 mM or less NaCl (e.g., about 50 to about 250 mM NaCl (e.g., about 250 mM NaCl)). In some embodiments, the first wash buffer applied to the chromatography support comprises about 20 mM Tris and about 250 mM NaCl.

In some embodiments, the method further comprises applying a second wash buffer to the chromatography support after applying the first wash buffer. In some embodiments, the method further comprises separating a flow through comprising the second wash buffer. In some embodiments, the second wash buffer applied to the chromatography support has a pH of about 4.5 to about 8.5 (e.g., about pH 5.2). In some embodiments, the second wash buffer applied to the chromatography support comprises one or more of glycine, acetic acid, citric acid, phosphate, sodium chloride (NaCl), calcium, magnesium, EDTA, NaOH, and Tris-HCl. In some embodiments, the second wash buffer applied to the chromatography support comprises about 10 to about 200 mM glycine and/or about 20 to about 300 mM acetic acid (e.g., about 50 mM glycine and about 100 mM acetic acid). In some embodiments, the second wash buffer applied to the chromatography support comprises about 100 to about 500 mM NaCl (e.g., about 175 mM NaCl).

In some embodiments, the method further comprises applying a third wash buffer to the chromatography support after applying the second wash buffer. In some embodiments, the method further comprises separating a flow through comprising the third wash buffer. In some embodiments, the third wash buffer applied to the chromatography support has a pH of about 4.5 to about 8.5 (e.g., about pH 7.2). In some embodiments, the third wash buffer applied to the chromatography support has a pH of about 4.5 to about 8.5 (e.g., about 7.2). In some embodiments, the third wash buffer applied to the chromatography support comprises one or more of glycine, acetic acid, citric acid, phosphate, sodium chloride (NaCl), calcium, magnesium, EDTA, and Tris-HCl. In some embodiments, the third wash buffer applied to the chromatography support comprises about 5 to about 100 mM Tris-HCl (e.g., about 20 mM Tris-HCl). In some embodiments, the first wash buffer applied to the chromatography support comprises about 400 mM or less NaCl (e.g., about 50 to about 250 mM NaCl (e.g., about 200 mM NaCl)). In some embodiments, the first wash buffer applied to the chromatography support comprises about 20 mM Tris and about 200 mM NaCl. In some embodiments, the elution buffer applied to the chromatography support has a pH of about 4.5 to about 8.5 (e.g., about pH 7.2). In some embodiments, the elution buffer applied to the chromatography support comprises one or more of glycine, acetic acid, citric acid, phosphate, sodium chloride (NaCl), calcium, magnesium, EDTA, and Tris-HCl. In some embodiments, the elution buffer applied to the chromatography support comprises about 5 to about 100 mM Tris-HCl (e.g., about 20 mM Tris-HCl). In some embodiments, the elution buffer applied to the chromatography support comprises about 1,000 or less mM NaCl (e.g., about 750 mM NaCl).

In some embodiments, the method further comprises applying a dilution buffer to the biological material prior to step (i). In some embodiments, the dilution buffer comprises deionized water. In some embodiments, the dilution buffer comprises purified water. In some embodiments, the dilution buffer has a pH of about 4.5 to about 8.5 (e.g., about pH 7.2). In some embodiments, the biological material is diluted 1:1 to 1:10 (v/v) with the dilution buffer. In some embodiments, the dilution buffer comprises one or more of glycine, acetic acid, citric acid, phosphate, sodium chloride (NaCl), calcium, magnesium, EDTA, and Tris-HCl. In some embodiments, the dilution buffer comprises about 5 to about 100 mM Tris-HCl (e.g., about 20 mM Tris-HCl). In some embodiments, the dilution buffer comprises about 300 mM NaCl or less (e.g., no salt or about 200 mM NaCl).

In some embodiments, the method further comprises detecting an amount of IαIp in the flow through of step (i). In some embodiments, the method comprises discarding the flow through of step (i).

In some embodiments, the method further comprises detecting an amount of IαIp in the eluate in step (ii).

In some embodiments, the method comprises a flow rate of about 1 to 10 mL/minute.

In some embodiments, the IαIp collected in the eluate of step (b) has a purity of about 5% to 99% or greater by weight relative to the purity of the IαIp in the biological material.

In some embodiments, the yield of IαIp in the eluate collected in step (b) is greater than about 20% (w/w) relative to the IαIp present in the biological material (e.g., 35% to about 90% or greater (e.g., about 95% or greater)). In some embodiments, the yield of IαIp from the biological material is at least about 5 μg/ml, about 50 μg/ml, about 100 μg/ml, about 300 μg/ml, about 600 μg/ml, or about 900 μg/ml (e.g., about 5 μg/ml to about 900 μg/ml).

In some embodiments, the purity of the IαIp is at least about 5% (w/w) (e.g., at least about 25%, about 50% (w/w), or about 75% (w/w) (e.g., from about 5% (w/w) to about 75% (w/w) or more (e.g., up to about 90%, 95%, 97%, 99%, or 100% (w/w)).

In some embodiments, the IαIp comprises two or more of inter-alpha inhibitor (IαI), pre-alpha inhibitor (PαI), and bikunin.

In some embodiments, the IαIp present in the biological material comprises between 60% to 80% (w/w) IαIp and/or between 20% to 40% (w/w) PαI; and/or wherein the IαIp present in the eluate of step (b) comprises between 60% to 80% (w/w) IαIp and/or between 20% to 40% (w/w) PαI.

In some embodiments, the IαIp comprises two or more of inter-alpha inhibitor (IαI), pre-alpha inhibitor (PαI), and bikunin. In some embodiments, the IαIp is inter-alpha inhibitor (IαI). In some embodiments, the IαIp is pre-alpha inhibitor (PαI). In some embodiments, the IαIp is (IαI) and pre-alpha inhibitor (PαI).

In some embodiments, the IαIp present in the biological material comprises between 60% to 80% (w/w) IαIp and/or between 20% to 40% (w/w) PαI; and/or wherein the IαIp present in the eluate of step (b) comprises between 60% to 80% (w/w) IαIp and/or between 20% to 40% (w/w) PαI.

In some embodiments, essentially all of the IαIp present in the biological material is IαI. In some embodiments, between about 90% and about 99.5% (w/w) of the IαIp present in the biological material is IαI. In some embodiments, between about 80% and about 90% (w/w) of the IαIp present in the biological material is IαI. In some embodiments, between about 70% and about 80% (w/w) of the IαIp present in the biological material is IαI. In some embodiments, between about 60% and about 70% (w/w) of the IαIp present in the biological material is IαI. In some embodiments, between about 50% and about 60% (w/w) of the IαIp present in the biological material is IαI. In some embodiments, between about 40% and about 50% (w/w) of the IαIp present in the biological material is IαI. In some embodiments, between about 30% and about 40% (w/w) of the IαIp present in the biological material is IαI. In some embodiments, between about 20% and about 30% (w/w) of the IαIp present in the biological material is IαI. In some embodiments, between about 10% and about 20% (w/w) of the IαIp present in the biological material is IαI. In some embodiments, between about 1% and about 10% (w/w) of the IαIp present in the biological material is IαI.

In some embodiments, essentially all of the IαIp present in the biological material is PαI. In some embodiments, between about 90% and about 99.5% (w/w) of the IαIp present in the biological material is PαI. In some embodiments, between about 80% and about 90% (w/w) of the IαIp present in the biological material is PαI. In some embodiments, between about 70% and about 80% (w/w) of the IαIp present in the biological material is PαI. In some embodiments, between about 60% and about 70% (w/w) of the IαIp present in the biological material is PαI. In some embodiments, between about 50% and about 60% (w/w) of the IαIp present in the biological material is PαI. In some embodiments, between about 40% and about 50% (w/w) of the IαIp present in the biological material is PαI. In some embodiments, between about 30% and about 40% (w/w) of the IαIp present in the biological material is PαI. In some embodiments, between about 20% and about 30% (w/w) of the IαIp present in the biological material is PαI. In some embodiments, between about 10% and about 20% (w/w) of the IαIp present in the biological material is PαI. In some embodiments, between about 1% and about 10% (w/w) of the IαIp present in the biological material is PαI.

In some embodiments, essentially all of the IαIp present in the biological material is IαI and PαI. In some embodiments, between about 90% and about 99.5% (w/w) of the IαIp present in the biological material is IαI and PαI. In some embodiments, between about 80% and about 90% (w/w) of the IαIp present in the biological material is IαI and PαI. In some embodiments, between about 95% and about 99.5% (w/w) of the IαIp present in the biological material is IαI and PαI. In some embodiments, between about 94% and about 98% (w/w) of the IαIp present in the biological material is IαI and PαI. In some embodiments, between about 93% and about 97% (w/w) of the IαIp present in the biological material is IαI and PαI. In some embodiments, between about 91% and about 96% (w/w) of the IαIp present in the biological material is IαI and PαI. In some embodiments, between about 90% and about 95% (w/w) of the IαIp present in the biological material is IαI and PαI. In some embodiments, between about 89% and about 94% (w/w) of the IαIp present in the biological material is IαI and PαI. In some embodiments, between about 88% and about 93% (w/w) of the IαIp present in the biological material is IαI and PαI. In some embodiments, between about 87% and about 92% (w/w) of the IαIp present in the biological material is IαI and PαI. In some embodiments, between about 86% and about 91% (w/w) of the IαIp present in the biological material is IαI and PαI. In some embodiments, between about 85% and about 90% (w/w) of the IαIp present in the biological material is IαI and PαI.

In some embodiments, essentially all of the IαIp present in the eluate of step (b) is IαI. In some embodiments, between about 90% and about 99.5% (w/w) of the IαIp present in the eluate of step (b) is IαI. In some embodiments, between about 80% and about 90% (w/w) of the IαIp present in the eluate of step (b) is IαI. In some embodiments, between about 70% and about 80% (w/w) of the IαIp present in the eluate of step (b) is IαI. In some embodiments, between about 68% and about 78% (w/w) of the IαIp present in the eluate of step (b) is IαI. In some embodiments, between about 66% and about 76% (w/w) of the IαIp present in the eluate of step (b) is IαI. In some embodiments, between about 64% and about 74% (w/w) of the IαIp present in the eluate of step (b) is IαI. In some embodiments, between about 62% and about 72% (w/w) of the IαIp present in the eluate of step (b) is IαI. In some embodiments, between about 60% and about 65% (w/w) of the IαIp present in the eluate of step (b) is IαI. In some embodiments, between about 61% and about 66% (w/w) of the IαIp present in the eluate of step (b) is IαI. In some embodiments, between about 62% and about 67% (w/w) of the IαIp present in the eluate of step (b) is IαI. In some embodiments, between about 63% and about 68% (w/w) of the IαIp present in the eluate of step (b) is IαI. In some embodiments, between about 64% and about 69% (w/w) of the IαIp present in the eluate of step (b) is IαI. In some embodiments, between about 65% and about 70% (w/w) of the IαIp present in the eluate of step (b) is IαI. In some embodiments, between about 66% and about 71% (w/w) of the IαIp present in the eluate of step (b) is IαI. In some embodiments, between about 67% and about 72% (w/w) of the IαIp present in the eluate of step (b) is IαI. In some embodiments, between about 68% and about 73% (w/w) of the IαIp present in the eluate of step (b) is IαI. In some embodiments, between about 69% and about 74% (w/w) of the IαIp present in the eluate of step (b) is IαI. In some embodiments, between about 59% and about 64% (w/w) of the IαIp present in the eluate of step (b) is IαI. In some embodiments, between about 58% and about 63% (w/w) of the IαIp present in the eluate of step (b) is IαI. In some embodiments, between about 57% and about 62% (w/w) of the IαIp present in the eluate of step (b) is IαI. In some embodiments, between about 56% and about 61% (w/w) of the IαIp present in the eluate of step (b) is IαI. In some embodiments, between about 55% and about 60% (w/w) of the IαIp present in the eluate of step (b) is IαI. In some embodiments, between about 54% and about 59% (w/w) of the IαIp present in the eluate of step (b) is IαI. In some embodiments, between about 54% and about 59% (w/w) of the IαIp present in the eluate of step (b) is IαI. In some embodiments, between about 53% and about 58% (w/w) of the IαIp present in the eluate of step (b) is IαI. In some embodiments, between about 52% and about 57% (w/w) of the IαIp present in the eluate of step (b) is IαI. In some embodiments, between about 51% and about 56% (w/w) of the IαIp present in the eluate of step (b) is IαI. In some embodiments, between about 50% and about 55% (w/w) of the IαIp present in the eluate of step (b) is IαI. In some embodiments, between about 49% and about 54% (w/w) of the IαIp present in the eluate of step (b) is IαI. In some embodiments, between about 48% and about 53% (w/w) of the IαIp present in the eluate of step (b) is IαI. In some embodiments, between about 47% and about 52% (w/w) of the IαIp present in the eluate of step (b) is IαI. In some embodiments, between about 46% and about 51% (w/w) of the IαIp present in the eluate of step (b) is IαI. In some embodiments, between about 45% and about 50% (w/w) of the IαIp present in the eluate of step (b) is IαI. In some embodiments, between about 60% and about 70% (w/w) of the IαIp present in the eluate of step (b) is IαI. In some embodiments, between about 58% and about 68% (w/w) of the IαIp present in the eluate of step (b) is IαI. In some embodiments, between about 56% and about 66% (w/w) of the IαIp present in the eluate of step (b) is IαI. In some embodiments, between about 54% and about 64% (w/w) of the IαIp present in the eluate of step (b) is IαI. In some embodiments, between about 52% and about 62% (w/w) of the IαIp present in the eluate of step (b) is IαI. In some embodiments, between about 50% and about 60% (w/w) of the IαIp present in the eluate of step (b) is IαI. In some embodiments, between about 48% and about 58% (w/w) of the IαIp present in the eluate of step (b) is IαI. In some embodiments, between about 46% and about 56% (w/w) of the IαIp present in the eluate of step (b) is IαI. In some embodiments, between about 44% and about 54% (w/w) of the IαIp present in the eluate of step (b) is IαI. In some embodiments, between about 42% and about 52% (w/w) of the IαIp present in the eluate of step (b) is IαI. In some embodiments, between about 40% and about 50% (w/w) of the IαIp present in the eluate of step (b) is IαI. In some embodiments, between about 30% and about 40% (w/w) of the IαIp present in the eluate of step (b) is IαI. In some embodiments, between about 20% and about 30% (w/w) of the IαIp present in the eluate of step (b) is IαI. In some embodiments, between about 10% and about 20% (w/w) of the IαIp present in the eluate of step (b) is IαI. In some embodiments, between about 1% and about 10% (w/w) of the IαIp present in the eluate of step (b) is IαI.

In some embodiments, essentially all of the IαIp present in the eluate of step (b) is PαI. In some embodiments, between about 90% and about 99.5% (w/w) of the IαIp present in the eluate of step (b) is PαI. In some embodiments, between about 80% and about 90% (w/w) of the IαIp present in the eluate of step (b) is PαI. In some embodiments, between about 70% and about 80% (w/w) of the IαIp present in the eluate of step (b) is PαI. In some embodiments, between about 60% and about 70% (w/w) of the IαIp present in the eluate of step (b) is PαI. In some embodiments, between about 50% and about 60% (w/w) of the IαIp present in the eluate of step (b) is PαI. In some embodiments, between about 40% and about 50% (w/w) of the IαIp present in the eluate of step (b) is PαI. In some embodiments, between about 38% and about 48% (w/w) of the IαIp present in the eluate of step (b) is PαI. In some embodiments, between about 36% and about 46% (w/w) of the IαIp present in the eluate of step (b) is PαI. In some embodiments, between about 34% and about 44% (w/w) of the IαIp present in the eluate of step (b) is PαI. In some embodiments, between about 32% and about 42% (w/w) of the IαIp present in the eluate of step (b) is PαI. In some embodiments, between about 30% and about 35% (w/w) of the IαIp present in the eluate of step (b) is PαI. In some embodiments, between about 31% and about 36% (w/w) of the IαIp present in the eluate of step (b) is PαI. In some embodiments, between about 32% and about 37% (w/w) of the IαIp present in the eluate of step (b) is PαI. In some embodiments, between about 33% and about 38% (w/w) of the IαIp present in the eluate of step (b) is PαI. In some embodiments, between about 34% and about 39% (w/w) of the IαIp present in the eluate of step (b) is PαI. In some embodiments, between about 35% and about 40% (w/w) of the IαIp present in the eluate of step (b) is PαI. In some embodiments, between about 30% and about 40% (w/w) of the IαIp present in the eluate of step (b) is PαI. In some embodiments, between about 28% and about 38% (w/w) of the IαIp present in the eluate of step (b) is PαI. In some embodiments, between about 26% and about 36% (w/w) of the IαIp present in the eluate of step (b) is PαI. In some embodiments, between about 24% and about 34% (w/w) of the IαIp present in the eluate of step (b) is PαI. In some embodiments, between about 22% and about 32% (w/w) of the IαIp present in the eluate of step (b) is PαI. In some embodiments, between about 30% and about 35% (w/w) of the IαIp present in the eluate of step (b) is PαI. In some embodiments, between about 29% and about 34% (w/w) of the IαIp present in the eluate of step (b) is PαI. In some embodiments, between about 28% and about 33% (w/w) of the IαIp present in the eluate of step (b) is PαI. In some embodiments, between about 27% and about 32% (w/w) of the IαIp present in the eluate of step (b) is PαI. In some embodiments, between about 26% and about 31% (w/w) of the IαIp present in the eluate of step (b) is PαI. In some embodiments, between about 25% and about 30% (w/w) of the IαIp present in the eluate of step (b) is PαI. In some embodiments, between about 24% and about 29% (w/w) of the IαIp present in the eluate of step (b) is PαI. In some embodiments, between about 23% and about 28% (w/w) of the IαIp present in the eluate of step (b) is PαI. In some embodiments, between about 22% and about 27% (w/w) of the IαIp present in the eluate of step (b) is PαI. In some embodiments, between about 21% and about 26% (w/w) of the IαIp present in the eluate of step (b) is PαI. In some embodiments, between about 20% and about 25% (w/w) of the IαIp present in the eluate of step (b) is PαI. In some embodiments, between about 20% and about 30% (w/w) of the IαIp present in the eluate of step (b) is PαI. In some embodiments, between about 10% and about 20% (w/w) of the IαIp present in the eluate of step (b) is PαI. In some embodiments, between about 1% and about 10% (w/w) of the IαIp present in the eluate of step (b) is PαI.

In some embodiments, essentially all of the IαIp present in the eluate of step (b) is IαI and PαI. In some embodiments, between about 90% and about 99.5% (w/w) of the IαIp present in the eluate of step (b) is IαI and PαI. In some embodiments, between about 80% and about 90% (w/w) of the IαIp present in the eluate of step (b) is IαI and PαI. In some embodiments, between about 95% and about 99.5% (w/w) of the IαIp present in the eluate of step (b) is IαI and PαI. In some embodiments, between about 94% and about 98% (w/w) of the IαIp present in the eluate of step (b) is IαI and PαI. In some embodiments, between about 93% and about 97% (w/w) of the IαIp present in the eluate of step (b) is IαI and PαI. In some embodiments, between about 91% and about 96% (w/w) of the IαIp present in the eluate of step (b) is IαI and PαI. In some embodiments, between about 90% and about 95% (w/w) of the IαIp present in the eluate of step (b) is IαI and PαI. In some embodiments, between about 89% and about 94% (w/w) of the IαIp present in the eluate of step (b) is IαI and PαI. In some embodiments, between about 88% and about 93% (w/w) of the IαIp present in the eluate of step (b) is IαI and PαI. In some embodiments, between about 87% and about 92% (w/w) of the IαIp present in the eluate of step (b) is IαI and PαI. In some embodiments, between about 86% and about 91% (w/w) of the IαIp present in the eluate of step (b) is IαI and PαI. In some embodiments, between about 85% and about 90% (w/w) of the IαIp present in the eluate of step (b) is IαI and PαI.

In some embodiments, the IαIp present in the eluate of step (b) comprises between about 60% to about 70% (w/w) IαI and between about 20% to 30% (w/w) PαI. In some embodiments, the IαIp present in the eluate of step (b) comprises between about 62% to about 72% (w/w) IαI and between about 28% to 30% (w/w) PαI.

In some embodiments, the IαIp has an apparent molecular weight of between about 60 to about 280 kDa.

In some embodiments, the IαIp has biological activity (e.g., cytokine inhibitor activity, chemokine inhibitor activity, or serine protease inhibitor activity).

In some embodiments, the biological material is a blood product material (e.g., whole plasma, cryo-poor plasma, liquid plasma, frozen plasma (FP) (e.g., fresh frozen plasma (FFP), FFP24, FP24, thawed FFP, thawed FFP24, thawed FP, thawed FP24, and a diluted or concentrated preparation thereof), source plasma, recovered plasma, solvent/detergent-treated plasma (SDP), platelet-rich plasma (PRP), platelet-poor plasma (PPP), serum, whole blood, and a diluted or concentrated preparation thereof).

In some embodiments, the biological material is a plasma fraction intermediate which is produced through one or more process steps (e.g. filtration, centrifugation, sedimentation, chromatography, adsorption, isolation, freezing, thawing, dilution, concentration, virus clearance, etc) from the blood product material.

In some embodiments, the biological material is a blood product which is produced through one or more process steps (e.g. filtration, centrifugation, sedimentation, chromatography, adsorption, isolation, freezing, thawing, dilution, concentration, virus clearance, etc) from the blood product material.

In some embodiments, the biological material is milk or colostrum.

In some embodiments, the biological material is from a mammal (e.g., a human, primate, bovine, equine, porcine, ovine, feline, or canine).

In some embodiments, the biological material is substantially unprocessed prior to application to the endotoxin-binding agent.

In some embodiments, the method further comprises performing one or more chromatography steps (e.g., repeating the method of the first aspect and/or its embodiments one or more times using the eluate collected in step (b)).

In some embodiments, the elution buffer applied to the endotoxin-binding agent in step (b) has a pH of 7.2 and comprises about 20 mM Tris-HCl and about 500 mM NaCl.

In some embodiments, the first wash buffer applied to the endotoxin-binding agent has a pH of 5.2 and comprises about 75 mM glycine, about 100 mM acetic acid, and about 150 mM NaCl.

In some embodiments, the second wash buffer applied to the endotoxin-binding agent has a pH of 7.2 and comprises about 20 mM Tris-HCl and about 300 mM NaCl.

In some embodiments, the elution buffer applied to the chromatography support has a pH of 7.2 and comprises about 20 mM Tris-HCl and about 750 mM NaCl.

In some embodiments, the first wash buffer applied to the chromatography support has a pH of 7.2 and comprises about 20 mM Tris-HCl and about 250 mM NaCl.

In some embodiments, the second wash buffer applied to the chromatography support has a pH of 5.2 and comprises about 50 mM glycine, about 100 mM acetic acid, and about 175 mM NaCl.

A second aspect of the disclosure features a composition comprising the IαIp produced by the method of the first aspect and its embodiments. In an embodiment, the composition is suitable for administration to a human.

A third aspect of the disclosure features a pharmaceutical composition comprising the composition of the second aspect and its embodiments and a pharmaceutically acceptable excipient.

A fourth aspect of the disclosure features a method of treating a disease or condition in a subject in need thereof by administering to the subject the composition of the second aspect or one of its embodiments or the pharmaceutical composition of the third aspect.

A fifth aspect of the disclosure features a kit comprising the composition of the second aspect or one of its embodiments or the pharmaceutical composition of the third aspect. In an embodiment, the kit further comprises instructions for therapeutic use.

A sixth aspect of the disclosure features a method of purifying an IαIp from plasma by: (a) diluting the plasma with a dilution buffer comprising deionized water to form diluted plasma; (b) applying the diluted plasma to an ETOXICLEAR™ resin and separating a flow through comprising the diluted plasma that does not bind to the ETOXICLEAR™ resin; (c) applying a first wash buffer comprising about 75 mM glycine, about 100 mM AcOH, and about 150 mM NaCl at a pH of about 5.2 to the ETOXICLEAR™ resin and separating a flow through comprising the first wash buffer; (d) applying a second wash buffer comprising about 20 mM Tris-HCl and about 300 mM NaCl at a pH of about 7.2 to the ETOXICLEAR™ resin and separating a flow through comprising the second wash buffer; and (e) applying an elution buffer comprising about 20 mM Tris-HCl and about 500 mM NaCl at a pH of 7.2 to the ETOXICLEAR™ resin and collecting an eluate comprising the IαIp.

A seventh aspect of the disclosure features a method of purifying an IαIp from plasma comprising: (a) diluting the plasma with a dilution buffer comprising 15 mM phosphate at a pH of about 5.5 to the plasma to form diluted plasma; (b) applying the diluted plasma to a DETOXI-GEL™ resin and separating a flow through comprising the diluted plasma that does not bind to the DETOXI-GEL™ resin; (c) applying a first wash buffer comprising about 15 mM phosphate and about 50 mM NaCl at a pH of about 5.5 to the DETOXI-GEL™ resin and separating a flow through comprising the first wash buffer; (d) applying a second wash buffer comprising about 15 mM phosphate and about 100 mM NaCl at a pH of about 5.5 to the DETOXI-GEL™ resin and separating a flow through comprising the second wash buffer; and (e) applying an elution buffer comprising about 15 mM phosphate and about 1,000 mM NaCl at a pH of about 5.5 to the DETOXI-GEL™ resin and collecting an eluate comprising the IαIp.

### Definitions

As used herein, the singular form “a,” “an,” and “the” includes plural references unless indicated otherwise.

As used herein, the term “about” means+/−10% of the recited value.

As used herein, “administering” means a method of giving a dosage of a substance (e.g., an IαIp) or a composition to a subject. The IaαIps utilized in the methods described herein can be administered, for example, orally, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraumbilically, intraocularly, topically, locally, by inhalation, by injection, by infusion, by continuous infusion, by localized perfusion bathing target cells directly, by catheter, by lavage, in creams, or in lipid compositions. The method of administration can vary depending on various factors (e.g., the substance or composition being administered and the severity of the condition, disease, or disorder being treated).

The term “biological material” as used herein refers to a sample from a subject (e.g., a mammal, such as a human) that contains IαIp. Examples of the biological material include a blood product material, e.g., whole plasma, cryo-poor plasma, liquid plasma, fresh frozen plasma (FFP), FFP24, frozen plasma (FP), FP24, thawed FFP, thawed FFP24, thawed FP, thawed FP24, source plasma, recovered plasma, solvent/detergent-treated plasma (SDP), platelet-rich plasma (PRP), platelet-poor plasma (PPP), serum, blood, and a diluted or concentrated preparation thereof, milk or colostrum, urine, sputum, and cerebrospinal fluid. Examples of the biological material include a plasma fraction intermediate or a blood product which is produced through one or more process steps (e.g. filtration, centrifugation, sedimentation, chromatography, adsorption, isolation, freezing, thawing, dilution, concentration, virus clearance, etc). The biological material can be from a human, primate, bovine, equine, porcine, ovine, feline, canine, or combinations thereof. The biological material may also be an extract prepared using cells that express IαIp, or may be or contain cells that secrete IαIp, e.g., cells that have been recombinantly modified to express IαIp.

The biological material can be substantially unprocessed, such that, prior to a purification step using, e.g., an endotoxin-binding agent, as described herein, no other purification step(s) has been applied to the material, or such that any prior purification step(s) performed with the material removes less than 10% (w/w) (e.g., less than 0.1%-10% (w/w), such as less than 0.1%, 0.5%, 1%, 2%, 3%, 4%, 5%, 6%, 7%, 8%, or 9% (w/w)) of one or more substances from the material. The biological material can also be one that has been processed or prepared prior to a purification step using, e.g., an endotoxin-binding agent, as described herein. For example, the biological materials could be processed using a decanting step, a clarification step, a chromatography step (e.g., anion exchange chromatography), a centrifugation and/or sedimentation step, a freezing step, a drying step, an evaporation step, an extraction step, a filtration step, a precipitation step, or by other purification or preparatory methods known in the art. The processing step may remove up to, e.g., 10% or more (w/w) (e.g., more than 10-30% (w/w), such as 15%, 20%, 25%, or 30% (w/w)) of one or more substances from the biological material (e.g., a protein other than an IαIp).

As used herein, the term “chromatography” or “chromatography step” refers to a separation of one or more analytes in a mixture by passing the mixture in a solution or in a suspension through a medium in which the analytes of the mixture move at different rates. For example, a chromatography step can be size exclusion chromatography, ion-exchange chromatography, affinity chromatography, or dye-ligand chromatography. More specifically, a chromatography step, as described herein, can be performed using, e.g., an endotoxin-binding agent (such as a resin) to separate an IαIp from a biological material containing the IαIp.

In this disclosure, the terms “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, the term “eluate” refers to a fraction containing an analyte material (e.g., IαIp) that is eluted from a medium (e.g., a support material) during a purification step (e.g., a chromatography step). An eluate may be released from the medium by applying an eluent to the medium, thereby releasing the analyte. More specifically, an eluate can refer to a fraction containing IαIp that has been released from a medium (e.g., an endotoxin-binding agent) following application of an eluent (e.g., an elution buffer, such as a buffer containing a salt) to the medium.

As used herein, the term “endotoxin” refers to a substance that includes a lipid and a polysaccharide. An endotoxin, such as a lipopolysaccharide (LPS), is typically found in the outer membrane of the cell wall of Gram-negative bacteria. An endotoxin can be approximately 10 kDa in size, but can readily form large aggregates of up to 1,000 kDa. LPS (or endotoxin) can be found in E. coli, as well as other gram negative bacteria (see, e.g., Bertani and Ruiz, EcoSal Plus doi:10.1128/ecosalplus.ESP-0001-2018, 2018, which is incorporated herein by reference in its entirety).

An “endotoxin-binding agent” as used herein is meant a molecule that can, or is known to, bind an endotoxin, such as a lipopolysaccharide. The endotoxin-binding agent may be one that specifically binds to an endotoxin. The endotoxin-binding agent can be, e.g., a resin that is known to be used to remove lipopolysaccharides from a biological material or a mixture of proteins. Exemplary endotoxin-binding agents include, but are not limited to, ETOXICLEAR™ (www.astreabioseparations.com/product/etoxiclear), PIERCE™ High Capacity Endotoxin Removal Resin (www.thermofisher.com/order/catalog/product/88270#/88270), TOXINERASER™ Endotoxin Removal Resin (www.genscript.com/kit/L00402-ToxinEraser sup_TM_sup_Endotoxin_Removal_Resin.html), PURKINE™ Endotoxin Removal Resin (www.abbkine.com/product/purkine-endotoxin-removal-resin-bmr21400/), DETOXI-GEL™ Endotoxin Removing Gel (www.thermofisher.com/order/catalog/product/20339 #/20339), and PROMEGA™ Endotoxin Removal Resin (www.promega.com/products/nucleic-acid-extraction/plasmid-purification/endotoxin-removal-resin/?catNum=A2191). The endotoxin-binding agent can be a molecule, e.g., polymyxin B, polylysine or a derivative thereof, or a synthetic mimetic peptide.

As used herein, the term “flow through” refers to a fraction of material or a volume of fluid that passes through a medium (e.g., a medium used for chromatography, such as an endotoxin-binding agent) without binding. Additional mobile phase (e.g., a fluid, such as buffer with a low (e.g., less than 50 mM salt) or no salt (e.g., sodium chloride)) can be added to ensure that one or more components of a mixture applied to a medium is fully loaded onto the medium, and to achieve initial or additional separation of an analyte (e.g., IαIp) from other components in the mixture.

As used herein, the terms “inter-alpha inhibitor protein” and “IαIp,” and plural forms thereof, refer to multi-component glycoproteins in a family of structurally related serine protease inhibitors. IaαIps have been shown to be important in the inhibition of an array of proteases including neutrophil elastase, plasmin, trypsin, chymotrypsin, Granzyme K, preprotein convertase, furin, cathepsin G, and acrosin. In human plasma, IaαIps are found at relatively high concentrations (400-800 mg/L). Unlike other inhibitor molecules, this family of inhibitors typically includes a combination of polypeptide chains (light and heavy chains) covalently linked by a chondroitin sulfate chain. The heavy chains of IaαIps (H1, H2, and H3) are also called hyaluronic acid (HA) binding proteins. The major forms of IaαIps found in human plasma are inter-alpha-inhibitor (IαI), which contains two heavy chains (H1 and H2) and a single light chain (L), and pre-alpha-inhibitor (PαI), which contains one heavy (H3) and one light chain (L). Another IαIp is the light chain (also termed bikunin (bi-kunitz inhibitor) with two Kunitz domains), which is known to broadly inhibit plasma serine proteases. Another IαIp is the heavy chain-related molecule H4, which circulates in the blood without linkage to bikunin. Yet another IαIp is the heavy chain-related molecule H5. IαI and PαI present in the plasma fraction have an apparent molecular weight of between about 60 kDa to about 280 kDa.

As used herein, the term “pharmaceutically acceptable excipient” means one or more compatible solid or liquid fillers, diluents, or encapsulating substances that are suitable for administration into a human. The excipient can contain an additive, such as a substance that enhances isotonicity and/or chemical stability. Such materials are non-toxic to recipients in the amounts and concentrations employed, and can include buffers, such as phosphate, citrate, succinate, acetate, lactate, tartrate, and other organic acids or their salts; tris-hydroxymethylaminomethane (Tris), bicarbonate, carbonate, and other organic bases and their salts; antioxidants, such as ascorbic acid; low molecular weight (for example, less than about ten residues) polypeptides, e.g., polyarginine, polylysine, polyglutamate and polyaspartate; proteins, such as serum albumin, gelatin, or immunoglobulins; hydrophilic polymers, such as polyvinylpyrrolidone (PVP), polypropylene glycols (PPGs), and polyethylene glycols (PEGs); amino acids, such as glycine, glutamic acid, aspartic acid, histidine, lysine, or arginine; monosaccharides, disaccharides, and other carbohydrates including cellulose or its derivatives, glucose, mannose, sucrose, dextrins or sulfated carbohydrate derivatives, such as heparin, chondroitin sulfate or dextran sulfate; polyvalent metal ions, such as divalent metal ions including calcium ions, magnesium ions and manganese ions; chelating agents, such as ethylenediamine tetraacetic acid (EDTA); sugar alcohols, such as mannitol or sorbitol; counterions, such as sodium or ammonium; and/or nonionic surfactants, such as polysorbates or poloxamers. Other additives may be also included, such as stabilizers, anti-microbials, inert gases, fluid and nutrient replenishers (i.e., Ringer's dextrose), electrolyte replenishers, and the like, which can be present in conventional amounts.

As used herein, the terms “prevent,” “preventing,” “prevention,” “prophylactic treatment” and the like refer to reducing the probability of developing a disorder or condition in a subject, who does not have, but is at risk of or is susceptible to developing, a disease, disorder, or condition.

As used herein, the term “processed” refers to a biological material that has been modified using one or more sample preparation steps (e.g., filtration, centrifugation, sedimentation, chromatography, etc.) prior to contacting the material to an endotoxin-binding agent according to the methods described herein. Other examples of processing include a decanting step, a clarification step, a freezing step, a drying step, an evaporation step, an extraction step, a filtration step, a precipitation step, or another purification or preparatory method known in the art. The processing step may remove up to, e.g., 10% or more (w/w) (e.g., 10-30% (w/w), such as 15%, 20%, 25%, or 30% (w/w) or more) of one or more substances from the biological material (e.g., a protein other than an IαIp).

As used herein, the terms “purify,” “purifying,” “purification” and the like refer to one or more steps or processes of removing proteins (e.g., proteins other than an IαIp) and/or non-proteinaceous substances (e.g., phospholipids and nucleic acids) from a heterologous mixture (e.g., a biological material, such as blood or milk) containing IαIp and the other proteins and/or substances to produce a composition containing an IαIp without the other proteins and/or substances present in the original mixture (e.g., a biological material) or in which the proteins other than IαIp and/or substances have been reduced by 40% or more by weight (e.g., 50%, 60%, 70%, 80%, 85%, 90%, 95%, 97%, or 99% or more) relative to, e.g., a starting mixture (e.g., a biological material). Examples of proteins that can be removed from a mixture containing an IαIp include, but are not limited to, alpha-1 antitrypsin, C1-inhibitor, albumin, a globulin (including immunoglobulins, e.g., IgA, IgG (e.g., of intravenous Ig (IVIg), anti-D IgG, hepatitis B IgG, measles IgG, rabies IgG, tetanus IgG, and Varicella Zoster IgG), IgM, IgD, and IgE), fibrinogen (factor I), prothrombin (factor II), thrombin, anti-thrombin III, factor III, factor V, factor VII, factor VIII, factor IX, factor X, factor XI, factor XII, factor XIII, fibronectin, alpha-2 antiplasmin, urokinase, protein C, protein S, protein Z, protein Z-related protease inhibitor, plasminogen, tissue plasminogen activator, plasminogen activator inhibitor-1, plasminogen activator inhibitor-2, von Willebrand factor, factor H, prekallikrein, high-molecular-weight kininogen, and heparin cofactor II.

As used herein, the term “pure” or “purity” refers to the extent to which an analyte has been isolated and is free of other components. In the context of proteins, purity of an isolated protein can be expressed with regard to the protein that is free of any contaminants (e.g., one or more unrelated proteins or other substances). For example, purity of an IαIp composition indicates how much of the composition is IαIp by total weight of the isolated material, which may be determined using, e.g., pure IαIp as a reference. A level of purity found in the disclosure can be 5%, 10%, 15%, 20%, 25%, 30%, 35%, 40%, 45%, 50%, 55%, 60%, 65%, 70%, 75%, 80%, 85%, 90%, 95%, greater than 95%, or greater than 99% (w/w). A “pure” IαIp composition of the disclosure can be greater than 20%, 25%, 30%, 35%, 40%, 45%, 50%, 55%, 60%, 65%, or up to 70% pure by weight. A “substantially pure” IαIp composition can be substantially free of contaminants or impurities, e.g., greater than 70%, 75%, 80%, 85%, 90%, 95%, or >99% purity by weight. In some embodiments, the level of contaminants or impurities is no more than about 20%, 15%, 12%, 10%, 9%, 8%, 7%, 6%, 5%, 4%, 3%, 2%, or 1% by weight. Purity can be determined by detecting a level of a specific analyte (e.g., IαIp) using an immunoassay or other technique (e.g., MAb 69.26—heparin-biotin sandwich ELISA, SDS/PAGE, and/or Western blot) and calculating a percentage of the analyte (w/w) relative to the total protein content (e.g., as determined by a total protein assay (e.g., bicinchoninic acid assay (BCA), Bradford assay, Biuret test, or another assay known in the art)).

As used herein, the term “subject” refers to a mammal, including, but not limited to, a human or non-human mammal, such as a primate, bovine, equine, porcine, ovine, feline, or canine. The subject may be a patient.

The term “substantially unprocessed,” as used herein, refers to a biological material that has been minimally modified, if at all, relative to the original source material (e.g., blood). For example, a substantially unprocessed biological material can retain the original content (e.g., the same proteins and/or substances and/or the same ratio of two or more proteins or substances) and/or the original characteristics (e.g., one or more biological activities) of the original source material. A biological material can be substantially unprocessed, such that any prior purification step(s) performed with the material removes less than 10% (w/w) (e.g., less than 0.1%-10% (w/w), such as less than 0.1%, 0.5%, 1%, 2%, 3%, 4%, 5%, 6%, 7%, 8%, or 9% (w/w)) of one or more proteins or substances from the material. A substantially unprocessed biological material may be one that has not been modified using a sample preparation step (e.g., filtration, centrifugation, sedimentation, chromatography, etc.), in particular, for example, prior to contacting the biological material to an endotoxin-binding agent according to the methods described herein.

As used herein, the phrase “specifically binds” refers to a binding reaction between an analyte (e.g., a protein, such as an IαIp) and a binding agent (e.g., an endotoxin-binding agent). A specific binding reaction is one that occurs between an analyte and a binding agent even in the presence of a heterogeneous population of proteins and other biological molecules (e.g., proteins other than IαIp in a biological material). Specific binding between an analyte (e.g., an IαIp) and a binding agent (e.g., an endotoxin-binding agent) can be characterized by a K_{d} of less than about 1000 nM (e.g., between 1 pM and 1000 nM). An analyte (e.g., an IαIp) that does not specifically bind to a binding agent (e.g., an endotoxin-binding agent) can be characterized by a K_{d} of greater than about 1000 nM (e.g., greater than 1 μM, 100 μM, 500 μM, or 1 mM).

As used herein, the term “support” means any apparatus that contains an agent (e.g., an endotoxin-binding agent) that can be contacted with a material (e.g., a biological material) containing at least one analyte (e.g., an IαIp). A support may be a column, a membrane, a disc, a chip, or other apparatus for chromatography or affinity capture, examples of which are known in the art and described herein.

As used herein, the term “treating” refers to reducing or ameliorating a disorder and/or one or more symptoms associated therewith. It will be appreciated that, although not precluded, treating a disorder or condition does not require that the disorder or symptoms associated therewith be completely eliminated.

As used herein, the term “yield” refers to the relative amount of an analyte (e.g., IαIp) obtained after a purification step or process as compared to the amount of analyte in the starting material (e.g., the biological material) (w/w). The yield may be expressed as a percentage. In the context of the disclosure, the amount of analyte (e.g., IαIp) in the starting material and analyte obtained after the purification step can be measured using an immunoassay or assay (e.g., an anti-IαIp antibody (e.g., MAb 69.26)-heparin-biotin sandwich ELISA, SDS/PAGE, and/or Western blot). The methods of the disclosure can be used to produce a yield of purified IαIp of about 20% (w/w) or greater relative to the amount present in the original biological material. For example, the methods can be used to produce a yield of purified IαIp of about 25%, 30%, 35%, 40%, 45%, 50%, 55%, 65%, 70%, 75%, 80%, 85%, or 90% (w/w) or greater.

### BRIEF DESCRIPTION OF THE DRAWINGS

FIGS. 1A and 1B are images showing the starting material, flow through, wash fractions, and eluted fraction obtained during the chromatographic isolation of IαIp from cryo-poor plasma. The starting material, flow through, wash fractions, and eluate were separated on a 4-15% Mini-PROTEAN® TGX Stain-Free (BioRad) SDS-PAGE gel (FIG. 1A) and transferred onto a nitrocellulose membrane for Western blot analysis (FIG. 1B). IαIp was detected using a monoclonal antibody against IαIp (Mab 69.26) and biotin-conjugated heparin. Both the 125 kDa and 250 kDa bands of IαIp, corresponding to PαI and IαI, respectively, were detected in the elution fraction of cryo-poor plasma using a Q anion-exchange resin (arrows in FIG. 1A, Lane 5) and the elution fraction of processed cryo-poor plasma using an endotoxin-binding agent (ETOXICLEAR™; arrows in FIG. 1A, Lane 10). Lanes 1-6 of the SDS-PAGE gel and Western blot correspond to the results of Q-anion-exchange chromatography using cryo-poor plasma as the starting material, while lanes 7-12 correspond to the results of purification with an endotoxin-binding agent using the eluate of the Q-anion-exchange chromatography as the starting material. The lanes are as follows: Lane 1: Starting material—cryo-poor plasma; Lane 2: Flow through from Q anion-exchange resin; Lane 3: Wash fraction 1 (pH 7.2, 250 mM NaCl); Lane 4: Wash fraction 2 (pH 5.2, 175 mM NaCl); Lane 5: Eluate from Q anion-exchange resin (pH 7.2, 750 mM NaCl); Lane 6: Cleaning from Q anion-exchange resin (1 M NaOH+2 M NaCl); Lane 7: Flow through from endotoxin-binding agent; Lane 8: Wash fraction 1 (pH 5.2, 150 mM NaCl); Lane 9: Wash fraction 2 (pH 7.2, 300 mM NaCl); Lane 10: Eluate fraction 1 (pH 7.2, 500 mM NaCl); Lane 11: Eluate fraction 2 (pH 7.2, 1,000 mM NaCl); Lane 12: Cleaning from endotoxin-binding agent (1 M NaOH+2 M NaCl).

FIG. 2 is a chromatogram showing the chromatographic isolation of IαIp from human plasma using an endotoxin-binding agent (DETOXI-GEL™) and 15 mM phosphate buffer (pH 7.3). IαIp was detected in the peaks indicated with an arrow. The y-axis shows absorbance (A280) and the x-axis shows time (minutes).

FIG. 3 is a chromatogram showing the chromatographic isolation of IαIp from human plasma using an endotoxin-binding agent (DETOXI-GEL™) and 15 mM phosphate buffer (pH 5.5). IαIp was detected in the peaks indicated with an arrow. The y-axis shows absorbance (A280) and the x-axis shows time (minutes).

FIG. 4 is a chromatogram showing the chromatographic isolation of IαIp from human plasma using an endotoxin-binding agent (DETOXI-GEL™) and 20 mM Tris-HCl buffer (pH 7.2). IαIp was detected in the peaks indicated with an arrow. The y-axis shows absorbance (A280) and the x-axis shows time (minutes).

FIGS. 5A and 5B are images showing the starting material, flow through, wash fractions, and eluted fraction obtained during the chromatographic isolation of IαIp from cryo-poor plasma. The starting material, flow through, wash fractions, and eluate were separated on a 4-20% TGX Stain-Free (BioRad) precast SDS-PAGE gel (FIG. 5A) and transferred onto a nitrocellulose membrane for Western blot analysis (FIG. 5B). IαIp was detected using a biotinylated monoclonal antibody against IαIp (Mab 69.26) and HRP-conjugated streptavidin. Both the 125 kDa and 250 kDa bands of IαIp, corresponding to PαI and IαI, respectively, were detected in the elution fraction of the cryo-poor plasma using a Q anion-exchange resin (arrows in FIG. 5B, Lane 5) and the elution fraction of the cryo-poor plasma using an endotoxin-binding agent (ETOXICLEAR™; arrows in FIG. 5B, Lane 6-7). Lanes 1-5 of the SDS-PAGE gel and Western blot correspond to the results of Q-anion-exchange chromatography using the cryo-poor plasma as the starting material, while lanes 6-7 correspond to the results of purification with an endotoxin-binding agent using the eluate of the Q-anion-exchange chromatography as the starting material. The lanes are as follows: Lane 1: Starting material—cryo-poor plasma; Lane 2: Flow through from Q anion-exchange resin; Lane 3: Wash fraction 1 (pH 7.2, 250 mM NaCl); Lane 4: Wash fraction 2 (pH 5.2, 150 mM NaCl); Lane 5: Eluate from Q anion-exchange resin (pH 7.2, 750 mM NaCl); Lane 6: Eluate fraction 1 (pH 7.2, 400 mM NaCl); Lane 7: Eluate fraction 2 (pH 7.2, 500 mM NaCl).

FIGS. 6A and 6B are images showing the starting material, flow through, wash fractions, and eluted fraction obtained during the chromatographic isolation of IαIp from the cryo-poor plasma. The starting material, flow through, wash fractions, and eluate were separated on a 4-20% TGX Stain-Free (BioRad) precast SDS-PAGE gel (FIG. 6A) and transferred onto a nitrocellulose membrane for Western blot analysis (FIG. 6B). IαIp was detected using a biotinylated monoclonal antibody against IαIp (Mab 69.26) and HRP-conjugated streptavidin. Both the 125 kDa and 250 kDa bands of IαIp, corresponding to PαI and IαI, respectively, were detected in the elution fraction of the cryo-poor plasma using a Q anion-exchange resin (arrows in FIG. 6B, Lane 5) and the elution fraction of the cryo-poor plasma using an endotoxin-binding agent (ETOXICLEAR™; arrows in FIG. 6B, Lane 9). Lanes 1-7 of the SDS-PAGE gel and Western blot correspond to the results of Q-anion-exchange chromatography using the cryo-poor plasma as the starting material, while lanes 8-12 correspond to the results of purification with an endotoxin-binding agent using the eluate of the Q-anion-exchange chromatography as the starting material. The lanes are as follows: Lane 1: Starting material—cryo-poor plasma; Lane 2: Flow through from Q anion-exchange resin; Lane 3: Wash fraction 1 (pH 7.2, 250 mM NaCl); Lane 4: Wash fraction 2 (pH 5.2, 150 mM NaCl); Lane 5: Eluate fraction 1 from Q anion-exchange resin (pH 7.2, 750 mM NaCl); Lane 6: Eluate fraction 2 from Q anion-exchange resin (pH 7.2, 1000 mM NaCl); Lane 7: Cleaning from Q anion-exchange resin (1 M NaOH+2 M NaCl); Lane 8: Flow through from endotoxin-binding agent and Wash fraction (pH 5.2, 150 mM NaCl); Lane 9: Eluate fraction 1 (pH 7.2, 500 mM NaCl); Lane 10: Eluate fraction 2 (pH 7.2, 1,000 mM NaCl); Lane 11: Cleaning from endotoxin-binding agent (1 M NaOH+2 M NaCl); Lane 12: Concentrated Eluate fraction 1 after buffer exchange and ultrafiltration (Millipore Ultracell 30 kDa cut off centrifugal membrane).

### DETAILED DESCRIPTION

The disclosure features methods of purifying an IαIp (e.g., IαI, PαI, a heavy chain (e.g., H1, H2, H3, H4, and/or H5), a light chain (e.g., bikunin), or a combination thereof) from a biological material (e.g., blood) by using an endotoxin-binding agent. The methods involve applying a biological material (e.g., a processed or substantially unprocessed biological material, such as blood or milk) containing IαIp(s) to an endotoxin-binding agent. We discovered that endotoxin-binding agents, such as endotoxin-specific chromatography resins (e.g., ETOXICLEAR™ and DETOXI-GEL™), bind IaαIps, which was not expected. Surprisingly, an endotoxin-binding agent, when used in the process of purifying an IαIp, reduces the loss of IαIp during purification process, thereby retaining or improving the yield of recovered IαIp, relative to other methods. In addition, the use of an endotoxin-binding agent as part of the IαIp purification process also maintains or increases the purity of the recovered IαIp.

Thus, we have used such endotoxin-binding agents to purify IaαIps, for example, from a biological source, such as blood. Moreover, a yield and purity of the IaαIps of up to 50% or more (e.g., up to 90% or more) could be obtained when using an endotoxin-binding agent as part of the purification process. This discovery can be used to simplify the purification process and concurrently increase the yield and purity of IαIps when employing the methods of the disclosure.

Also featured are pharmaceutical compositions prepared using the purified IαIps obtained by the methods described herein and methods for treating and/or reducing the likelihood of developing a disease or condition in a subject in need thereof by administering a pharmaceutical composition prepared using the purified IαIps obtained by the methods described herein.

### Methods of Purification

An endotoxin-binding agent can be used to purify an IαIp (e.g., IαI, PαI, a heavy chain (e.g., H1, H2, H3, H4, and/or H5), a light chain (e.g., bikunin), or a combination thereof) from a biological material (e.g., blood and milk). The methods described below can be used to separate IαIp from other components present in the biological material. The methods can be used to prepare IαIps with a purity ranging from about 5% to about 99% or greater (e.g., 5%, 10%, 15%, 20%, 25%, 30%, 35%, 40%, 45%, 50%, 55%, 60%, 65%, 70%, 75%, 80%, 85%, 90%, 95%, greater than 95%, such as 97% or 99%, or greater than 99%). In addition, the methods can be used to produce a yield of purified IαIp of about 20% (w/w) or greater relative to the amount present in the original biological material. For example, the methods can be used to produce a yield of purified IαIp of about 20%, 25%, 30%, 35%, 40%, 45%, 50%, 55%, 65%, 70%, 75%, 80%, 85%, or 90% (w/w) or greater.

Biological Material

The methods described below can be used to purify IαIp from a biological material. The biological material containing IαIp can be obtained from a human, primate, bovine, equine, porcine, ovine, feline, canine, or combinations thereof. The biological material can be, e.g., blood, milk (e.g., colostrum), urine, sputum, and cerebrospinal fluid. For example, the biological material can be, but is not limited to, whole plasma, cryo-poor plasma, liquid plasma, fresh frozen plasma (FFP), FFP24, frozen plasma (FP), FP24, thawed FFP, thawed FFP24, thawed FP, thawed FP24, source plasma, recovered plasma, solvent/detergent-treated plasma (SDP), platelet-rich plasma (PRP), platelet-poor plasma (PPP), serum, blood (e.g., whole blood), and a diluted or concentrated preparation thereof. The biological material containing IαIp can be, but is not limited to, a plasma fraction intermediate. The plasma fraction intermediate is produced through one or more process steps (e.g. filtration, centrifugation, sedimentation, chromatography, adsorption, isolation, freezing, thawing, dilution, concentration, S/D treatment, etc) from whole plasma, cryo-poor plasma, liquid plasma, fresh frozen plasma (FFP), FFP24, frozen plasma (FP), FP24, thawed FFP, thawed FFP24, thawed FP, thawed FP24, source plasma, recovered plasma, solvent/detergent-treated plasma (SDP), platelet-rich plasma (PRP), platelet-poor plasma (PPP), serum, blood (e.g., whole blood), and a diluted or concentrated preparation thereof.

The biological material may also be an extract prepared using cells expressing IαIp or may be or contain cells that secrete IαIp, e.g., recombinant cells that have been modified to express IαIp.

The biological material may contain, in addition to IαIp, a mixture of proteins, such as three or more proteins found in the blood or three or more proteins found in milk (e.g., colostrum). For instance, the biological material may contain alpha-1 antitrypsin, C1-inhibitor, albumin, a globulin (such as an immunoglobulin, e.g., IgA, IgG (e.g., intravenous Ig (IVIg), anti-D IgG, hepatitis B IgG, measles IgG, rabies IgG, tetanus IgG, and Varicella Zoster IgG), IgM, IgD, and IgE), fibrinogen (factor I), prothrombin (factor II), thrombin, anti-thrombin III, factor III, factor V, factor VII, factor VIII, factor IX, factor X, factor XI, factor XII, factor XIII, fibronectin, alpha-2 antiplasmin, urokinase, protein C, protein S, protein Z, protein Z-related protease inhibitor, plasminogen, tissue plasminogen activator, plasminogen activator inhibitor-1, plasminogen activator inhibitor-2, von Willebrand factor, factor H, prekallikrein, high-molecular-weight kininogen, and heparin cofactor II. The biological material may be milk (e.g., colostrum), which may contain one or more of whey (e.g., up to about 50-80% (w/w); e.g., beta-lactoglobulin (e.g., about 1-5% (w/w)), alpha-lactalbumin (e.g., about 0.5-2% (w/w)), albumin, ovalbumin, and a globulin (e.g., an immunoglobulin, such as IgA, IgG (e.g., IVIg), IgM, IgD, and IgE, e.g., about 0.01-1% (w/w)), casein (e.g., alpha-casein and/or beta-casein; e.g., up to about 3-35% (w/w)), lactoferrin (e.g., about 0.01-0.2% (w/w), lactose, alpha-1 antitrypsin, anti-chymotrypsin, plasminogen, fibrinogen, growth factors, and cytokines).

The biological material contacted or applied to an endotoxin-binding agent (or to a different support described herein, such as an anion-exchange chromatography support), according to the methods described below, can be substantially unprocessed (e.g., original source material) or the biological material can be processed prior to being contacted or applied to the endotoxin-binding agent, for example, by using one or more sample preparation methods or other known purification methods, such as those described herein.

A substantially unprocessed biological material is one that has been minimally modified, if at all, relative to the original source material (e.g., blood or another source, as described herein), such that the biological material maintains the original characteristics of the source material. For example, a substantially unprocessed biological material may be subjected to a sample preparation or purification step(s) that removes less than 10% (w/w) (e.g., less than 0.1%-10% (w/w), such as less than 0.1%, 0.5%, 1%, 2%, 3%, 4%, 5%, 6%, 7%, 8%, or 9% (w/w)) of one or more substances from the material before the material is contacted or applied to an endotoxin-binding agent. Alternatively, a substantially unprocessed biological material can be an eluate or a fraction from a prior purification step(s), in which the prior purification step(s) removes less than 10% of impurities from the material.

The biological material may also be subjected to one or more processing steps, such as those described herein, prior to application of the biological material to an endotoxin-binding agent (or to a different support described herein, such as an anion-exchange chromatography support). For example, one or more proteins found in the biological material (e.g., blood or milk), such as, e.g., alpha-1 antitrypsin, C1-inhibitor, albumin, a globulin (such as an immunoglobulin, e.g., IgA, IgG (e.g., intravenous Ig (IVIg), anti-D IgG, hepatitis B IgG, measles IgG, rabies IgG, tetanus IgG, and Varicella Zoster IgG), IgM, IgD, and IgE), fibrinogen (factor I), prothrombin (factor II), thrombin, anti-thrombin III, factor III, factor V, factor VII, factor VIII, factor IX, factor X, factor XI, factor XII, factor XIII, fibronectin, alpha-2 antiplasmin, urokinase, protein C, protein S, protein Z, protein Z-related protease inhibitor, plasminogen, tissue plasminogen activator, plasminogen activator inhibitor-1, plasminogen activator inhibitor-2, von Willebrand factor, factor H, prekallikrein, high-molecular-weight kininogen, and heparin cofactor II can be partially removed (e.g., 1%-70% (w/w) removed) or substantially removed (e.g., greater than 70%-100% (w/w) removed) from the biological material prior to a purification step using an endotoxin-binding agent.

Methods of the disclosure can be used to prepare IaαIps (e.g., IαI, PαI, a heavy chain (e.g., H1, H2, H3, H4, and/or H5), a light chain (e.g., bikunin), or a combination thereof) from a biological material containing three to ten (or more) of the aforementioned blood proteins. The disclosed methods can also be used to separate IaαIps from a biological material containing three to fifteen, three to twenty, three to twenty five, three to thirty, ten to twenty, ten to twenty five, ten to thirty, fifteen to twenty five, fifteen to thirty, twenty to thirty, or thirty or more different proteins (e.g., blood or milk proteins). The biological material applied to an endotoxin-binding agent may also contain, in addition to IαIp, about 40-65% (e.g., about 55%) albumin by total weight of protein in the biological material, about 25-45% (e.g., about 38%) globulins by total weight of protein, about 2-12% (e.g., about 7%) fibrinogen by total weight of protein, or any combination thereof, by total weight of protein.

Endotoxin-Binding Agent

The methods described herein involve the use of an endotoxin-binding agent, which can be used to bind to IαIp in a mixture of proteins (e.g., IαIp present in a biological material, such as milk or blood). An endotoxin-binding agent is a molecule that may be known to bind to an endotoxin, such as a lipopolysaccharide. The endotoxin-binding agent may be one that specifically binds to an endotoxin. The endotoxin-binding agent may be, for example, incorporated into or immobilized on a support. The support may be a monolithic support or a particle-based support. The particle can be, for example, a resin. The endotoxin-binding agent can be packed or immobilized on any number of known supports, e.g., a column, membrane, disc, or chip. The endotoxin-binding agent can be a molecule, e.g., polymyxin B, polylysine or a derivative thereof, or a synthetic mimetic peptide.

Non-limiting examples of the endotoxin-binding agent that can be used in the methods described herein include, e.g., ETOXICLEAR™, PIERCE™ High Capacity Endotoxin Removal Resin, TOXINERASER™ Endotoxin Removal Resin, PURKINE™ Endotoxin Removal Resin, DETOXI-GEL™ Endotoxin Removal Gel, or Promega™ Endotoxin Removal Resin. The methods can be performed using, e.g., a pre-packed column or cartridge containing the endotoxin-binding agent (e.g., a column having a volume of about 0.1 mL to about 100 mL, or a column having a larger volume).

A column containing an endotoxin-binding agent can be prepared, e.g., by applying a slurry of endotoxin-binding agent suspended in buffer (e.g., deionized water) to a filter-fritted column (e.g., a column of about 2 to about 100 mL, or larger) and allowing the endotoxin-binding agent to settle for about 30 minutes. The settled resin can then be equilibrated with about 3 to about 5 column volumes of a suitable, pyrogen-free buffer or water (e.g., deionized water) before a biological material is applied.

In some examples, DETOXI-GEL™ Endotoxin Removal Gel is used as the source of the endotoxin-binding agent. DETOXI-GEL™ uses immobilized polymyxin B to bind lipid A domains of endotoxins. In another example, ETOXICLEAR™ is used as the endotoxin-binding agent.

Reagents for Use in the Purification Methods

Dilution Buffer

A biological material containing IαIp (e.g., IαI, PαI) may be combined with a dilution buffer prior to a purification step, such as prior to contacting or applying the biological material to a medium, such as an endotoxin-binding agent. The dilution buffer can be added to lower a salt (e.g., NaCl) concentration of the biological material, e.g., to avoid or reduce the possibility of early elution of IαIp off the endotoxin-binding agent. The biological material may be diluted, e.g., 1:1 to 1:10 (v/v) (e.g., 1:1, 1:2, 1:3, 1:4, 1:5, 1:6, 1:7, 1:8, 1:9, or 1:10 (v/v), such as 1:3 (v/v)) with the dilution buffer and then contacted or applied to the medium (e.g., an endotoxin-binding agent).

The dilution buffer can have a pH range of about 4.5 to 8.5 (e.g., about 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or about 8.5). The dilution buffer can contain one or more of deionized water, glycine, acetic acid, citric acid, phosphate, sodium chloride (NaCl), calcium, magnesium, EDTA, and Tris-HCl. A dilution buffer used for loading the biological material may have a low concentration of a salt, such as NaCl (e.g., 300 mM or less salt (e.g., NaCl, such as 200 mM, 150 mM, 100 mM, 75 mM, 50 mM, 25 mM, 10 mM, 5 mM, or 0 mM salt (e.g., NaCl)). For example, a biological material containing an IαIp can be diluted 1:3 (v/v) with a buffer containing 20 mM Tris-HCl and the diluted material can be contacted or applied to an endotoxin-binding column. The biological material can be, e.g., one that was prepared during a prior purification step (e.g., a chromatography step, such as a step using an anion-exchange support). The dilution buffer can be water.

Loading Buffer

A biological material containing IαIp (e.g., IαI, PαI) may be adjusted pH and conductivity prior to a purification step, such as prior to contacting or applying the biological material to a medium, such as an endotoxin-binding agent, anion exchanger. A loading buffer containing the biological material may have a pH range of about 4.5 to 8.5 (e.g., about 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or about 8.5). The loading buffer can contain one or more of deionized water, glycine, acetic acid, citric acid, phosphate, sodium chloride (NaCl), calcium, magnesium, EDTA, and Tris-HCl. The loading buffer may have a low concentration of a salt, such as NaCl (e.g., 300 mM or less salt (e.g., NaCl, such as 200 mM, 150 mM, 100 mM, 75 mM, 50 mM, 25 mM, 10 mM, 5 mM, or 0 mM salt (e.g., NaCl)). The buffer may contain about 20 mM Tris-HCl and 200 mM NaCl. The loading buffer can be, e.g., one that was prepared during a prior process step (e.g., a chromatography step, such as a step using an anion-exchange support, a filtration step). The loading buffer may have the conductivity of about 10 mS/cm to about 30 mS/cm, about 15 mS/cm to about 25 mS/cm, or about 20 mS/cm.

Flow Through Buffer

A flow through buffer can optionally be used to ensure that all of the biological material is loaded onto a medium (e.g., an endotoxin-binding agent or another support described herein) during a purification step. The flow through buffer can also be used to achieve initial or additional separation of the components present in the biological material following application to the medium. The flow through buffer can be the same as the dilution buffer. Alternatively, the flow through buffer can be different from the dilution buffer.

For example, a flow through buffer may have the same or a different pH (e.g., a pH within the range of about 4.5 to about 8.5 (e.g., about 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or about 8.5)) and/or a different constitution of components (e.g., about 5 mM to about 300 mM of one or more of glycine, acetic acid, citric acid, phosphate, NaCl, calcium, magnesium, EDTA, and Tris-HCl) relative to a dilution buffer used for loading a biological material onto the medium. A flow through buffer may have a higher salt concentration than that of a dilution buffer (e.g., a salt (e.g., NaCl) concentration of 5 to about 300 mM higher than that of the dilution buffer (e.g., the method may involve the use of a flow through buffer with about 150 mM NaCl following the use of a dilution buffer with less than 150 mM NaCl, such as a dilution buffer with no NaCl). The properties of the flow through buffer can be selected to improve initial separation among the components of the biological material during a purification step (e.g., during a column chromatography step).

Wash Buffer

The purification method can also include a wash buffer that is used in one or more wash steps (e.g., 1, 2, 3, 4, or more wash steps) that occur, e.g., after contacting or applying a biological material to an endotoxin-binding agent or during one or more other purification steps, as described herein. The wash step(s) can be performed to remove components (e.g., proteins or other substances found in the biological material that are not IαIp) that may present in, but less strongly bound to (e.g., weakly bound, such as with a K_{d} of about 1 mM or greater), the endotoxin-binding agent or other medium (e.g. anion exchanger).

The wash buffer applied to a medium (e.g., an endotoxin-binding agent or other type of support described herein) can be used to change the pH of the medium, to change the salt concentration of the medium, or to change both the pH and the salt concentration of the medium. A first wash buffer applied to the medium may change the pH, while a second wash buffer applied to the medium may change the salt concentration, or vice versa. The wash step(s) using a wash buffer can facilitate the purification of IαIp by promoting the release of proteins other than IαIp from the medium.

The wash buffer may differ from the flow through buffer, the dilution buffer or the loading buffer in terms of the components or other properties (e.g., pH, conductivity or salt concentration). For example, a wash buffer may contain a higher concentration of a salt (e.g., NaCl) than the concentration of a salt in the flow through or dilution buffer. If the salt concentration in the wash buffer is the same as the flow through or dilution buffer, the wash buffer may differ instead in its pH or in one or more of its components. For example, a flow through buffer used in the purification method may contain 20 mM Tris-HCl+150 mM NaCl (pH 7.2), whereas a wash buffer may contain 75 mM glycine+100 mM acetic acid+150 mM NaCl (pH 5.2). For example, a loading buffer used in the purification method may contain about 18 mM Tris-HCl+about 2 mM Tris+about 200 mM NaCl (about pH 7.2), whereas a first wash buffer contains about 18 mM Tris-HCl+about 2 mM Tris+about 250 mM NaCl (pH 7.2), whereas a second wash buffer may contain about 75 mM Glycine+about 100 mM HAc+about 150 mM NaCl+about 92.5 mM NaOH (about pH 5.2). For example, a loading buffer used in the purification method may contain about about 18 mM Tris-HCl+about 2 mM Tris+about 200 mM NaCl (about pH 7.2), whereas a first wash buffer contains about 75 mM Glycine+about 100 mM HAc+about 200 mM NaCl (about pH 5.2), whereas a second wash buffer may contain (about 18 mM Tris-HCl+about 2 mM Tris+about 250 mM NaCl (about pH 7.2).

The wash buffer may have a pH in the range of about 4.5 to about 8.5 (e.g., about 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or about 8.5). The wash buffer may have a pH of about 5.2. The wash buffer may have a pH of about 7.2. The wash buffer can also contain about 5 mM to about 400 mM of one or more of glycine, acetic acid, citric acid, phosphate, sodium chloride (NaCl), calcium, magnesium, EDTA, and Tris-HCl. For example, the wash buffer may have a concentration of a salt (e.g., NaCl) concentration of about 50 to about 400 mM. For example, the wash buffer may have a concentration of a salt (e.g., NaCl) concentration of about 200 to about 300 mM. For example, the wash buffer may have a concentration of a salt (e.g., NaCl) concentration of about 250 mM, about 250 mM, or about 300 mM. For example, the wash buffer may have about 200 to about 300 mM NaCl. For example, the wash buffer may have about 200 mM NaCl, about 250 mM NaCl or about 300 mM NaCl. The wash buffer may also be prepared with a pH that differs from other buffers previously used in the purification process (e.g., a dilution buffer, flow through buffer, and/or prior wash buffer(s)).

Wash buffers used in the purification methods may contain different salt concentrations and may be applied to a medium, such as an endotoxin-binding agent, an anion exchange resin, starting with a low salt concentration wash buffer followed by a subsequent wash step(s) using a wash buffer(s) with an increasing salt concentration at each wash step.

For example, a first wash step may involve applying a first wash buffer with a low pH (e.g., less than pH 7.0, such as pH 5.5 or less (e.g., pH 5.2)) and/or salt concentration (e.g., a salt (e.g., NaCl) concentration of less than 150 mM) to a medium, such as an endotoxin-binding agent (e.g., a first wash buffer may contain 75 mM glycine+100 mM acetic acid+150 mM NaCl (pH 5.2)) and a second wash step may involve applying a second wash buffer with a higher pH (e.g., a pH above pH 7.0, such as pH 7.2) and/or salt concentration (e.g., a salt (e.g., NaCl) concentration of greater than 150 mM (e.g., about 300 mM)) to the medium (e.g., a second wash buffer may contain 20 mM Tris-HCl+300 mM NaCl (pH 7.2)). In another example, a first wash step may use a wash buffer containing 15 mM phosphate+50 mM NaCl (pH 5.5), whereas a subsequent wash step may use a second wash buffer containing 15 mM phosphate+100 mM NaCl (pH 5.5).

Elution Buffer

The purification method may also include the collection of an eluate containing IαIp from a medium (e.g., an endotoxin-binding agent or other agent, such as an anion exchange resin). The method involves contacting or applying an eluent or elution buffer to the medium (e.g., an endotoxin-binding agent, an anion exchange resin) and collecting the eluate.

The elution buffer may be prepared with a sufficiently high salt (e.g., sodium chloride (NaCl)) concentration (e.g., greater than about 200 mM salt (e.g., NaCl) (e.g., 250 mM, 300 mM, 350 mM, 375 mM, 400 mM, 450 mM, 500 mM, 550 mM, 600 mM, 650 mM, 700 mM, 750 mM, 800 mM, 850 mM, 900 mM, 950 mM, or 1,000 mM salt (e.g., NaCl)), such that bound IαIp can be released from the medium (e.g., an endotoxin-binding agent, anion exchange resin). The elution buffer may contain the same components as the buffers previously described (e.g., dilution buffer, loading buffer, flow through buffer, and wash buffer(s)) or the elution buffer may contain one or more different components (e.g., the elution buffer may contain about 5 mM to about 300 mM of one or more of glycine, acetic acid, citric acid, phosphate, and Tris-HCl). Other salts or additives can be used in place of NaCl, e.g., calcium, magnesium, or EDTA at an equivalent concentration. The pH of the elution buffer may also be the same as the buffers (e.g., dilution and/or wash buffer) previously contacted or applied to the medium (e.g., an endotoxin-binding agent) or the pH of the elution buffer may be different (e.g., the elution buffer may have a pH in the range of about 4.5 to about 8.5 (e.g., about 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or about 8.5)). The salt (e.g., NaCl) concentration in the elution buffer may be higher than the salt (e.g., NaCl) concentration in the wash buffer(s) previously applied to the medium (e.g., an endotoxin-binding agent). The pH of the elution buffer may be about 7.2. For example, the elution buffer may contain about 400 mM to about 1,000 mM NaCl. For example, the elution buffer may contain about 500 mM NaCl, about 750 mM NaCl, or 1000 mM NaCl. In a preferred example, IαIp is eluted from an endotoxin-binding agent by applying a buffer to the endotoxin-binding agent containing 20 mM Tris-HCl+500 mM NaCl (pH 7.2). Application of an elution buffer to the endotoxin-binding agent can be repeated one or more time. The elution buffer applied to the endotoxin-binding agent may be the same or different. For example, a second or subsequent elution buffer applied to the endotoxin-binding agent can contain a higher salt concentration (e.g., 1,000 mM NaCl) relative to a first or prior elution buffer (e.g., 500 mM NaCl). If desired, the fractions collected following application of the elution buffer can be analyzed separately for the presence and concentration of IaαIps using, e.g., ELISA or other techniques known in the art, and then, optionally pooled. Alternatively, the fractions may be pooled and then analyzed for the presence and concentration of IaαIps.

Cleaning Buffer

The purification method can also include an optional cleaning step, in which a cleaning buffer is applied to the medium in order to regenerate the medium (e.g., an endotoxin-binding agent) for use in another round of purification. The cleaning buffer can be prepared with a sufficiently high pH (e.g., about 12 to about 14). The cleaning buffer can contain about 1 M of an alkaline solute, e.g., sodium hydroxide (NaOH). Optionally, the cleaning buffer can additionally contain about 1 to about 2 M salt (e.g., NaCl). In a preferred example, the cleaning buffer contains 1 M NaOH+2 M NaCl (pH 14).

Purification of IαIp Using an Endotoxin-Binding Agent

IαIp can be purified from a biological material by contacting or applying the biological material containing the IαIp to an endotoxin-binding agent (e.g., one or more of the endotoxin-binding agents described herein). The biological material can be applied directly to the endotoxin-binding agent without dilution or, alternatively, the biological material can be diluted with a dilution buffer (as described above) and then applied to the endotoxin-binding agent. For example, the volume of a biological material (with or without dilution) contacted or applied to the endotoxin-binding agent may be, e.g., about 0.5 to about 20 column volumes (or other suitable volume).

After application of the biological material containing IαIp to the endotoxin-binding agent, the flow through can optionally be analyzed to confirm that it does not contain (or contains less than 10% (w/w)) IαIp. For example, an ELISA assay (e.g., using an anti-IαIp antibody, such as MAb 69.26) or other known techniques (e.g., SDS-PAGE, and/or Western Blot, or other known techniques) can be performed. Once the flow through has been confirmed to contain no or an insubstantial amount of IαIp (e.g., an amount of about 30 μg/mL or less, such as about 20 μg/mL, 10 μg/mL, 5 μg/mL, or 1 μg/mL or less), the flow through can be discarded. An additional flow through buffer may be applied (e.g., in a volume of about 1 to about 50 column volumes) to the endotoxin-binding agent to ensure that all of the biological material is loaded onto the endotoxin-binding agent. This flow through may then be discarded (e.g., after confirming the absence (or an insubstantial amount) of IαIp in the flow through, if desired).

Next, one or more wash steps (e.g., 2, 3, 4, or more wash steps) may be performed to remove non-IαIps present in the biological material that are weakly bound to the endotoxin-binding agent. About 0.5 to about 10 column volumes of a wash buffer (or other appropriate volume) can be applied in a given wash step. The resulting wash fractions can be analyzed (e.g., using ELISA or other known techniques), if desired, to confirm that it does not contain (or contains less than, e.g., 10% (w/w)) IαIp. If a substantial amount of IαIp (e.g., 10% (w/w) or greater) is detected in the wash buffer flow through, the flow through can, if desired, be processed to collect and purify the IαIp present in the flow through (e.g., using an endotoxin-binding agent or other medium described herein (e.g., an anion-exchange support)).

After the wash step(s) is performed, an eluate containing IαIp can be collected by applying an elution buffer (e.g., about 0.4 to about 5 column volumes or other suitable volume) to the endotoxin-binding agent. If desired, more than one (e.g., 2, 3, 4, or more) elution buffers may be applied to the endotoxin-binding agent to elute or to ensure elution of all IaαIps. The eluate can be analyzed for the presence of IaαIps using, e.g., ELISA or other techniques known in the art. The fractions from different elution buffers may then be pooled, if desired.

After an eluate is collected, the endotoxin-binding agent can optionally be cleaned by applying a cleaning buffer (e.g., about 0.5 to about 5 column volumes or other suitable volume) to the endotoxin-binding agent to regenerate the endotoxin-binding agent for a future use. The resulting cleaning fraction(s) can be analyzed (e.g., using ELISA or other known techniques), if desired, for the presence of IαIp.

Each step of the purification process (e.g., the application of the biological material, the wash step(s), and the elution step(s)) can be performed using either gravity flow or with low pressure (e.g., 0-15 psi) in order to produce a flow rate of about 1 to about 10 mL per minute (e.g., about 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 mL per minute). In some examples, the flow rate is about 2 mL per minute.

This process can be repeated one or more times, if desired. Alternatively, the IαIp can be further processed from the eluate fraction using techniques known in the art (e.g., concentration, dialysis, and/or lyophilization, among other techniques). Alternatively, the eluate fraction containing IαIp may be subjected to one or more additional purification steps, if desired, as is discussed below.

Purification of IαIp Following the Use of an Endotoxin-Binding Agent

An eluate fraction containing IαIp collected following elution from an endotoxin-binding agent can be further purified using other known purification steps (e.g., one or more of the steps described below under “Additional purification steps”). For example, the eluate containing IαIp can be subjected to one or more purification step(s), such as those described in, e.g., US 2003/0190732, US 2011/0190194, US 2012/0053113, and US 2014/0206844, each of which is incorporated herein by reference. Furthermore, the eluate containing IαIp can be further purified using an anion-exchange chromatography as described above.

Purification Step(s) Prior to the Use of an Endotoxin-Binding Agent

A biological material containing IαIp may be processed prior to contacting or applying the biological material to an endotoxin-binding agent. For example, the biological material may be processed using a sample preparation or purification step(s) that removes up to, e.g., 10% or more (w/w) (e.g., 10-30% (w/w), such as 15%, 20%, 25%, or 30% (w/w), or more) of one or more substances from the biological material (e.g., a protein or substance other than an IαIp).

Processing steps may include, for example, filtration, centrifugation, sedimentation, chromatography, decanting step, clarification, freezing, drying, evaporation, extraction, filtration, precipitation, or another purification or preparatory method known in the art. In a preferred example, the method involves performing anion-exchange chromatography (discussed below) using a biological material containing IαIp and, subsequently, contacting or applying an eluate containing one or more IaαIps prepared from the anion-exchange chromatography to an endotoxin-binding agent for further purification of the IaαIps as discussed above and herein.

A prior purification step or processing step can involve one or more of the purification methods described in, e.g., US 2003/0190732, US 2011/0190194, US 2012/0053113, and US 2014/0206844, each of which is incorporated herein by reference.

Additional Purification Steps

A biological material (e.g., cryo-poor plasma, plasma fraction intermediate, blood product) containing IαIp (e.g., IαI, PαI, a heavy chain (e.g., H1, H2, H3, H4, and/or H5), a light chain (e.g., bikunin), or a combination thereof) can be applied to one or more chromatography supports other than one containing an endotoxin-binding agent, either before or after purification of IαIp using an endotoxin-binding agent (e.g., by using an anion-exchange chromatography support). The chromatography support can be a monolithic or particle-based support. The chromatography support can be a column, membrane, disc, or chip. Additionally, the chromatography support can be, e.g., a size-exclusion chromatography support, an ion-exchange chromatography support, an affinity chromatography support, or a combination thereof.

For example, the monolithic support or particle-based support may contain an immobilized anion-exchange resin. The immobilized anion-exchange resin can be, e.g., a diethylaminoethane (DEAE) or a quaternary amine (Q) (e.g., Tosoh TOYOPEARL® GigaCap Q650M).

The biological material containing IαIp can be applied directly to the chromatography support (e.g., an anion-exchange chromatography support). Alternatively, the biological material may be diluted with a dilution buffer, as described above, and then applied to the support. For example, cryo-poor plasma can be diluted 1:3 (v/v) with dilution buffer containing, e.g., 20 mM Tris-HCl+200 mM NaCl (pH 7.2), and applied to the chromatography support (e.g., in a volume of about 0.5 to about 25 column volumes or other appropriate volume).

After applying the biological material to the chromatography support, the flow through can be separated and, optionally, analyzed to confirm that it does not contain (or contains less than 10% (w/w)) IαIp. For example, an ELISA assay or other known techniques (e.g., SDS-PAGE, and/or Western Blot, or other known techniques) can be performed. If a substantial amount of IαIp (e.g., 10% (w/w) or greater) is detected in the flow through, the flow through can, if desired, be processed to collect and purify the IαIp present in the flow through (e.g., using an endotoxin-binding agent or other medium described herein (e.g., an anion-exchange support)). Once the flow through has been confirmed to contain no or an insubstantial amount of IαIp (e.g., an amount of about 30 μg/mL or less, such as about 20 μg/mL, 10 μg/mL, 5 μg/mL, or 1 μg/mL or less), the flow through can be discarded.

Optionally, a flow through buffer can be prepared and applied (e.g., in a volume of about 1 to about 50 column volumes or other appropriate volume) to the chromatography support to ensure that all of the biological material is loaded onto the support. This flow through may then be discarded (e.g., after confirming the absence (or an insubstantial amount) of IαIp in the flow through, if desired).

Next, one or more wash steps (e.g., 2, 3, 4, or more) may be performed to remove non-IαIps of the biological material that are weakly bound to the chromatography support. The wash buffers may be prepared as described above. About 0.4 to about 10 column volumes of a wash buffer (or other appropriate volume) can be applied to the chromatography support in a given wash step. The resulting wash fractions can be analyzed (e.g., using ELISA or other known techniques), if desired, to confirm that it does not contain (or contains less than, e.g., 10% (w/w)) IαIp. If a substantial amount of IαIp (e.g., 10% (w/w) or greater) is detected in the wash buffer flow through, the flow through can, if desired, be processed to collect and purify the IαIp present in the flow through (e.g., using an endotoxin-binding agent or other medium described herein (e.g., an anion-exchange support)).

In an example, a first wash step is performed by applying a buffer containing 20 mM Tris-HCl+250 mM NaCl (pH 7.2) to the chromatography support (e.g., a Tosoh TOYOPEARL® GigaCap Q650M column), e.g., in a volume of about 5-10, such as about 8 or 9, column volumes, followed by a second wash step that is performed by applying a buffer containing 50 mM glycine+100 mM acetic acid+175 mM NaCl (pH 5.2) to the support, e.g., in a volume of about 2-6, such as about 4 or 5, column volumes.

A processed material containing IaαIps can then be obtained by applying an eluent or elution buffer (e.g., in a volume of about 0.4 to about 10 column volumes or other appropriate volume) to the chromatography support and collecting the eluate containing IaαIps. The elution buffer can be prepared and applied to the chromatography support in an analogous fashion as described above in connection with the endotoxin-binding agent purification process. If desired, more than one (e.g., 2, 3, 4, or more) elution buffers may be applied to the chromatography support to elute or to ensure elution of all IaαIps. The eluate can be analyzed for the presence of IaαIps using, e.g., ELISA (or other techniques known in the art). The fractions from different elution buffers may then be pooled, if desired.

In an example, an elution buffer containing 20 mM Tris-HCl+750 mM NaCl (pH 7.2) is applied to the chromatography support in a volume of, e.g., about 2-5 column volumes, such as, e.g., 3-4 column volumes. The eluate containing IaαIps can then be further processed by application to an endotoxin-binding agent, as described above, or the eluate can be further processed by repeating the chromatography step using the same or a different chromatography support, or by using one or more different purification or preparation steps, for example, filtration, centrifugation, sedimentation, chromatography, decanting step, clarification, freezing, drying, evaporation, extraction, filtration, precipitation, or another purification or preparatory method known in the art.

If desired, the chromatography support can be washed with a cleaning buffer to regenerate the chromatography support for a future use. The resulting cleaning fractions can be analyzed (e.g., using ELISA or other known techniques), if desired, for the presence of IαIp.

In an example, a cleaning buffer containing 1 M NaOH+2 M NaCl (pH 14) is applied to the chromatography support in a volume of, e.g., about 1-5 column volumes, such as, e.g., 1.5-2 column volumes.

Detection of IαIp

IαIp isolated using the purification methods described herein can be quantified using one or more assays known in the art, such as those described in, e.g., WO 2009/154695, US 2020/0057077, and WO 2020/086879, which are incorporated herein by reference.

For example, an IαIp quantification assay may involve contacting a sample containing IαIp to an IαIp binding agent (e.g., an antibody that specifically binds to IαIp (such as, e.g., MAb 69.26 (see, e.g., Sha et al., J Pediatr. 180:135-140, 2017), MAb 69.31 (see, e.g., Lim et al., J Infect Dis. 188(6):919-926, 2003), or PAb R22C (see, e.g., WO 2020/086879), each of which is herein incorporated by reference in its entirety) or an IαIp ligand (e.g., heparin, LPS, and/or hyaluronic acid (HA)) and detecting the amount of bound IαIp (e.g., using a detection agent). The IαIp binding agent may be labeled with, e.g., biotin, and detection can be by use of, e.g., horseradish peroxidase-labeled streptavidin, which can then be detected using known detection methods. In another method, the IαIp binding agent is labeled with, e.g., a fluorophore, which can be detected by spectrophotometry. Alternatively, the IαIp detection agent can be directly detected without the use of a label (e.g., by surface plasmon resonance (SPR)). After the addition of the IαIp detection agent, an additional wash step (e.g., one or more) can be performed to remove unbound IαIp detection agent.

IαIp can then be measured based on signal from the conjugated label or the bound detection agent (e.g., enzyme activity or fluorescence) using standard techniques known in the art. If an enzyme is used as the label, substrate can be added to produce the signal (for example, a color change) and can be read by a device suitable for detecting the signal, such as a spectrophotometer. The signal (for example, absorbance or fluorescence) can be plotted against a standard with known concentration of IαIp to establish a standard curve or can be compared against a known reference concentration. The unknown concentration in the samples can be calculated and determined based on the established standard curve or reference concentration value.

Isolated IαIp The purification methods described herein (e.g., purification using an endotoxin-binding agent, either alone or in combination with one or more additional pre- or post-purification steps, such as anion-exchange chromatography) can be used to purify IαIp (e.g., IαI, PαI, a heavy chain (e.g., H1, H2, H3, H4, and/or H5), a light chain (e.g., bikunin), or a combination thereof) from a biological material. A purity of the IαIps can be determined by detecting the total amount of IαIp obtained after the purification (e.g., using an assay or immunoassay such as MAb 69.26—heparin-biotin sandwich ELISA, SDS/PAGE, and/or Western blot) and calculating a percentage (w/w) relative to the total protein content determined by a total protein assay (e.g., bicinchoninic acid assay (BCA), Bradford assay, Biuret test, or another assay known in the art). After the purification step(s), the IαIp may have a purity of at least about 5% (w/w), e.g., at least about 5%, 10%, 15%, 20%, 25%, 30%, 35%, 40%, 45%, 50%, 55%, 60%, 65%, 70%, 75%, 80%, 85%, 90%, 95%, or >99% (w/w).

Isolated IaαIps have an apparent molecular weight of between about 60 kDa to about 280 kDa, which can be determined by any appropriate method known in the art, e.g., by sodium dodecyl sulfate polyacrylamide gel electrophoresis. The isolated IaαIps can also be tested for biological activity, such as, e.g., an activity selected from the group consisting of a cytokine inhibitor activity, chemokine inhibitor activity, protease inhibitor activity (e.g., serine protease inhibitor activity), chondroitin sulfate binding, glycosaminoglygan binding activity, hyaluronic acid binding activity, complement binding activity, histone binding activity, Arg-Gly-Asp (RGD) domain binding activity, coagulation factor binding activity, cellular repair activity, and extracellular matrix protein binding activity. The IαIp can also be tested for trypsin inhibitory specific activity, e.g., trypsin inhibitory specific activity between about 1000 IU/mg to about 2000 IU/mg (e.g., 1000, 1100, 1200, 1300, 1400, 1500, 1600, 1700, 1800, 1900, or 2000 IU/mg).

The proportion or concentration of IaαIps (e.g., IαI and/or PαI) present in a final purified fraction can vary. Preferably the purified IaαIps (e.g., IαI and/or PαI) are present in the final purified fraction in a physiological proportion. Physiological proportions may be, for example, the proportions found in a person or animal that is healthy and/or the ratio of IαI and PαI that appears naturally in human plasma. Physiological proportions are typically from between about 60% to about 80% IαI and between about 20% to about 40% PαI.

The purification methods also produce a yield of isolated IαIp of about 20% (w/w) or greater (e.g., about 25%, 30%, 35%, 40%, 45%, 50%, 55%, 60%, 65%, 70%, 75%, 80%, 85%, or 90% (w/w) or greater) relative to the IαIp present in the biological material (e.g., blood or milk). In some examples, the yield can be greater than or equal to about 90% or 95% by weight.

The disclosed purification methods can be used to produce a composition containing IαIp. The composition can also be prepared with a higher concentration of IαIp relative to the concentration of IαIp that was present in the original biological material (e.g., blood or milk). Thus, the methods can be used to prepare a composition containing IαIp in an amount of at least about 5 μg/mL, e.g., about 5 μg, 50 μg, 100 μg, 300 μg, 600 μg, 900 μg, 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, or 50 mg or more IαIp per mL. The IαIp present in the composition may make up greater than about 5 to about 99% or greater (w/w) of the composition (e.g., greater than 5, 10, 20, 30, 40, 50, 60, 70, 80, 90, 95, 97, 99% or greater (w/w)).

Additional steps (e.g., lyophilization, concentration in-vacuo) can be performed to increase the concentration of IαIp in a composition that is prepared using the disclosed methods. These known methods can be used, for example, to produce a composition containing IαIp in an amount of, e.g., about 1-50 mg/mL (e.g., about 1, 2, 3, 4, 5, 6, 7, 8, 9, or 10 mg or more IαIp per mL) from the IαIp purified according to the methods described herein.

### Pharmaceutical Compositions

An IαIp (e.g., IαI, PαI, a heavy chain (e.g., H1, H2, H3, H4, and/or H5), a light chain (e.g., bikunin), or a combination thereof) obtained by purification using the purification methods disclosed herein (e.g., using an endotoxin-binding agent, as described above) can be used to prepare a pharmaceutical composition. The IαIp can be combined, for example, with a pharmaceutically acceptable excipient. The pharmaceutical composition containing IαIp would be suitable for administration to a human.

Examples, of pharmaceutical compositions containing IαIp are described in, e.g., US 2007/0297982, US 2015/0238578, US 2019/0269765, and WO 2020/086879, which are herein incorporated by reference.

### Methods of Treatment

IαIp prepared by the methods described herein can be used (e.g., when prepared as a pharmaceutical composition) to treat or prevent various diseases, conditions, or symptoms thereof, e.g., diseases or conditions characterized by inflammation and/or low levels of an IαIp. Methods of treatment and methods of identifying a subject suitable for treatment with a pharmaceutical composition of the disclosure can be found in, e.g., US 2007/0297982, US 2015/0238578, US 2019/0269765, US 2020/0057077, and WO 2020/086879, which are herein incorporated by reference.

### Kits

IαIp prepared by the methods described herein can be used to prepare a kit containing the IαIp (e.g., IαI, PαI, a heavy chain (e.g., H1, H2, H3, H4, and/or H5), a light chain (e.g., bikunin), or a combination thereof). For example, the IαIp can be placed in a vial in an amount of about 0.5 to about 500 mg/mL (e.g., about 1-50 mg/mL) for distribution in the kit. Exemplary kits can be prepared as described in, e.g., US 2007/0297982, US 2015/0238578, US 2019/0269765, US 2020/0057077, and WO 2020/086879, which are herein incorporated by reference.

The following examples are intended to illustrate, rather than limit, the invention.

### Examples

### Example 1. Purification of an IαIp from Cryo-Poor Plasma Using ETOXICLEAR™

A sample of cryo-poor plasma was diluted (1:3 (v/v) dilution in 20 mM Tris-HCl+200 mM NaCl, pH 7.2) and the diluted cryo-poor plasma (114.4 mL) was applied to a commercially available 5 mL Q anion-exchange resin (Tosoh TOYOPEARL® GigaCap Q650M) at a flow rate of 3.5 mL per minute. The flow through was collected for analysis. The flow through, which did not contain (or contained an insubstantial amount of) IαIp was discarded. Additional plasma dilution buffer (172.7 mL: 20 mM Tris-HCl+200 mM NaCl, pH 7.2) was applied to the column to allow the starting material to pass through the column completely. The additional flow through was collected for analysis and was determined to contain no (or an insubstantial amount of) IαIp and was discarded.

The column was washed with a first wash buffer with approximately 8 CV (43.5 mL: 20 mM Tris-HCl+250 mM NaCl, pH 7.2) and the resulting fractions were collected. The combined fractions were analyzed for total protein (bicinchoninic acid assay (BCA)), IαIp (MAb 69.26—heparin-biotin sandwich ELISA), and trypsin inhibition activity as shown in Table 1. The combined wash fractions contained a small amount of impure IαIp (0.1% pure, 61.727 μg, 0.8761% yield of IαIp (w/w)) were discarded.

After the wash at pH 7.2, the column was further washed (approximately 5CV) with a second wash buffer with a lower pH (23.7 mL: 50 mM Gly+100 mM AcOH+175 mM NaCl, pH 5.2) and the resulting fractions were collected, combined, and analyzed as shown in Table 1. The combined wash fractions contained a small amount of impure IαIp (0.4% pure, 72.806 μg, 1.0339% yield of IαIp (w/w)) were discarded.

After the wash at pH 5.2, the bound protein was eluted with a high salt elution buffer (16.1 mL: 20 mM Tris-HCl+750 mM NaCl, pH 7.2). The fractions were collected, combined, and analyzed (e.g., total protein, IαIp, and trypsin inhibition activity) as shown in Table 1. The collected fractions contained enriched IαIp (56.43% pure, 98.537% yield of IαIp (w/w)).

A cleaning buffer with a high pH/high salt content (8.3 mL: 1 M NaOH+2 M NaCl) was next applied to the column and the resulting fractions were combined and analyzed for IαIp as described in Table 1. The combined fractions were determined to contain no (or an insubstantial amount of) IαIp and were discarded.

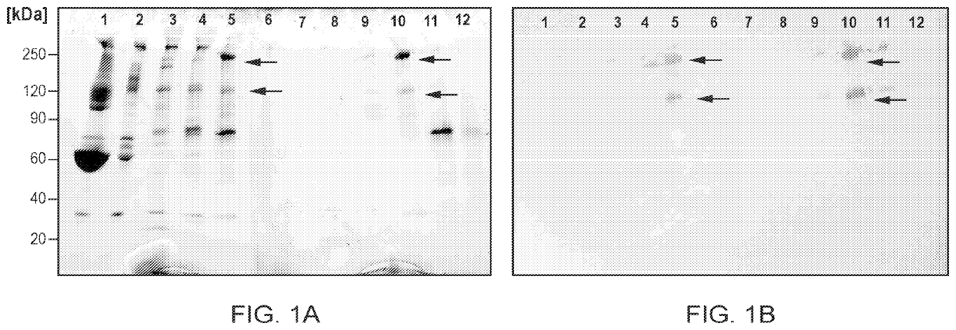

Table 1 provides a summary of the combined fractions and quantities of protein observed in each step (e.g., flow through, first wash, second wash, elution, and cleaning). Furthermore, FIG. 1A-1B show an SDS-PAGE and a Western blot, respectively, containing combined fractions from each step collected from the Tosoh TOYOPEARL® GigaCap Q650M column. The first step of the purification removed 98.6% of the initial plasma protein.

TABLE 1
Capture step using Q anion-exchange resin.

| | | | | IαIp ELISA | | | | | |
|---|---|---|---|---|---|---|---|---|---|
| | | Protein Assay | | (Heparin- | | Biological Activity | | | |
| | Vol. | (BCA) | | Sandwich) | | Trypsin Inhibition Activity | | | Purity |
| Fraction | (mL) | (μg/mL) | (μg) | (μg/mL) | (μg) | (IU/mL) | (IU) | (IU/mg) | (%) |
| Diluted | 114.4 | 7965.63 | 911268.30 | 61.59 | 7045.38 | 1889.14 | 216117.85 | 237.16 | 0.77 |
| cryo-poor | | | | | | | | | |
| plasma | | | | | | | | | |
| Flow | 172.7 | 4501.87 | 777472.77 | 0 | 0 | 1129.68 | 195096.60 | 250.94 | 0 |
| through | | | | | | | | | |
| Wash 1 | 43.5 | 839.17 | 36503.89 | 1.42 | 61.72 | 132.74 | 5774.02 | 158.17 | 0.1 |
| (pH 7.2) | | | | | | | | | |
| Wash 2 | 23.7 | 695.23 | 16477.05 | 3.07 | 72.81 | 126.38 | 2995.11 | 181.78 | 0.4 |
| (pH 5.2) | | | | | | | | | |
| Eluate | 16.1 | 764.15 | 12302.88 | 431.20 | 6942.32 | 617.66 | 9944.39 | 808.29 | 56.43 |
| Cleaning | 8.3 | 108.87 | 903.64 | 0 | 0 | 0 | 0 | 0 | 0 |
| | | Mass | 92.58% | Mass | 100.45% | Mass | 103.68% | | |
| | | Balance | | Balance | | Balance | | | |

Step Yield: 98.537%

The eluate from the Q anion-exchange column (16 mL) containing IαIp was then diluted (1:3 (v/v)) in dH₂O to a volume of 64.0 mL. This fraction was applied to a commercially available 32 mL column containing an endotoxin-binding agent (ETOXICLEAR™, Astrea Bioseparations) at a flow rate of 3.5 mL per minute. The flow through was collected for analysis and then discarded because no (or an insubstantial amount of) IαIp was detected. An additional buffer volume (26.7 mL: 20 mM Tris-HCl+150 mM NaCl, pH 7.2) was applied to the column. The total flow through volume was collected (90.7 mL) for analysis and then discarded as no (or an insubstantial amount of) IαIp was detected.

The column was washed with a first wash buffer with approximately 1.5 CV (47.0 mL: 75 mM Gly+100 mM AcOH+150 mM NaCl, pH 5.2) and the resulting fractions were collected. The fractions were combined and analyzed for total protein (bicinchoninic acid assay (BCA)), IαIp (69.26 Mab−heparin-biotin sandwich ELISA), and trypsin inhibition activity as shown in Table 2. The combined wash fractions were then discarded as no (or an insubstantial amount of) IαIp was detected.

The column was further washed with a second wash buffer having a higher pH (47.5 mL: 20 mM Tris-HCl+300 mM NaCl, pH 7.2) and the resulting fractions were collected and combined. The combined wash fractions contained a small amount of impure IαIp (0.53% pure, 39.045 μg, 0.5624% yield of IαIp (w/w) from the Q eluate) were discarded.

The bound protein was then eluted from the column by applying a high salt elution buffer (32.3 mL: 20 mM Tris-HCl+500 mM NaCl, pH 7.2). The resulting fractions were collected, combined, and analyzed as shown in Table 2. The combined eluate fractions were determined to contain highly pure IαIp (>99.9% pure, 99.5% yield (for the current step), 98.0% overall yield for two steps).

A second elution buffer (28.9 mL: 20 mM Tris-HCl+1000 mM NaCl, pH 7.2) was applied to the column to remove any remaining IαIp from the endotoxin-binding agent. No additional (or an insubstantial amount of) IαIp was detected in this eluate (see Table 2).

The column was cleaned by applying a cleaning buffer (18.2 mL: 1 M NaOH+2M NaCl); no additional (or an insubstantial amount of) IαIp was detected in the flow through.

Table 2 provides a summary of the combined fractions and quantities of protein observed in each step (e.g., flow through, first wash, second wash, eluate, second eluate, and cleaning). Furthermore, FIG. 1A-B show the results of testing by SDS-PAGE and Western blot of combined fractions for each step collected from the ETOXICLEAR™ column.

TABLE 2
Purification using ETOXICLEAR ™, pH 7.2.

| | | | | IαIp ELISA | | | | | |
|---|---|---|---|---|---|---|---|---|---|
| | | Protein Assay | | (Heparin- | | Biological Activity | | | |
| | Vol. | (BCA) | | Sandwich) | | Trypsin Inhibition Activity | | | Purity |
| Fraction | (mL) | (μg/mL) | (μg) | (μg/mL) | (μg) | (IU/mL) | (IU) | (IU/mg) | (%) |
| Diluted | 64.0 | 192.22 | 12302.88 | 108.47 | 6942.32 | 155.381 | 9944.390 | 808.298 | 56.43 |
| fraction | | | | | | | | | |
| from prior | | | | | | | | | |
| step | | | | | | | | | |
| Flow | 90.7 | 32.49 | 2947.29 | 0.000 | 0.000 | 0.000 | 0.000 | 0.000 | 0 |
| through | | | | | | | | | |
| Wash 1 | 47.0 | 49.65 | 2333.74 | 0.000 | 0.000 | 0.000 | 0.000 | 0.000 | 0 |
| (pH 5.2) | | | | | | | | | |
| Wash 2 | 47.5 | 53.45 | 2538.97 | 0.82 | 39.04 | 0.000 | 0.000 | 0.000 | 0.53 |
| (pH 7.2) | | | | | | | | | |
| Eluate 1 | 32.3 | 211.89 | 6844.27 | 213.79 | 6905.39 | 308.718 | 9971.591 | 1456.925 | >99.9 |
| Eluate 2 | 28.9 | 151.36 | 4374.51 | 0.000 | 0.000 | 0.000 | 0.000 | 0.000 | 0 |
| Cleaning | 18.2 | 71.95 | 1309.44 | 0.000 | 0.000 | 0.000 | 0.000 | 0.000 | 0.000 |
| | | Mass | 92.58% | Mass | 100.45% | Mass | 103.676% | | |
| | | Balance | | Balance | | Balance | | | |

Step Yield: 99.468%
Overall Yield: 98.013%

This example demonstrates that an endotoxin-binding agent can be used to selectively bind IαIps. This binding property can be used to purify IαIps from a biological material, such as blood (e.g., cryo-poor plasma).

### Example 2. Purification of an IαIp from Human Plasma Using DETOXI-GEL™, pH 7.3

A sample of human plasma (fresh frozen plasma, 2.5 mL) was diluted (1:3 (v/v) dilution in 15 mM phosphate, pH 7.3), and the diluted plasma (10.0 mL) was applied to a commercially available 4 mL column containing an endotoxin-binding agent (DETOXI-GEL™, Pierce) at a flow rate of 2 mL per minute.

The flow through was collected for analysis. Additional dilution buffer (50.2 mL: 15 mM phosphate, pH 7.3) was applied to the column to allow the starting material to pass through the column completely. The additional flow through was collected for analysis (see Table 3) and was determined to contain IαIp (0.159% pure, 108.281 μg, 14.87% yield of IαIp (w/w)).

The column was washed with a first wash buffer (24.6 mL: 15 mM phosphate+50 mM NaCl, pH 7.3) and the fractions were collected, combined, and analyzed for total protein (BCA assay) and IαIp (MAb 69.26—heparin-biotin sandwich ELISA). The combined fractions were determined to contain IαIp (1.390% pure, 179.186 μg, 24.61% yield of IαIp (w/w)).

After the 50 mM NaCl wash, the column was further washed with a second wash buffer having a higher concentration of NaCl (16.7 mL: 15 mM phosphate+100 mM NaCl, pH 7.3) and the resulting fractions were collected, combined and analyzed (see Table 3, Wash 2). The combined fractions were determined to contain a higher amount of IαIp than the previous wash step (4.969% pure, 235.353 μg, 32.33% yield of IαIp (w/w)).

After the 100 mM NaCl second wash, an elution buffer having a higher concentration of NaCl (7.3 mL: 15 mM phosphate+1,000 mM NaCl, pH 7.3) was applied to the endotoxin-binding agent. The resulting fractions were collected, combined, and analyzed (see Table 3, Wash 3). The combined fractions were determined to contain a lower amount of IαIp than the previous first or second wash steps (2.763% pure, 40.632 μg, 5.58% yield of IαIp (w/w)).

The column was further cleaned by washing with a high pH buffer (6.7 mL: 1 M NaOH) and the resulting fractions were collected, combined, and analyzed for IαIp (65.292 μg, 8.97% yield of IαIp (w/w)).

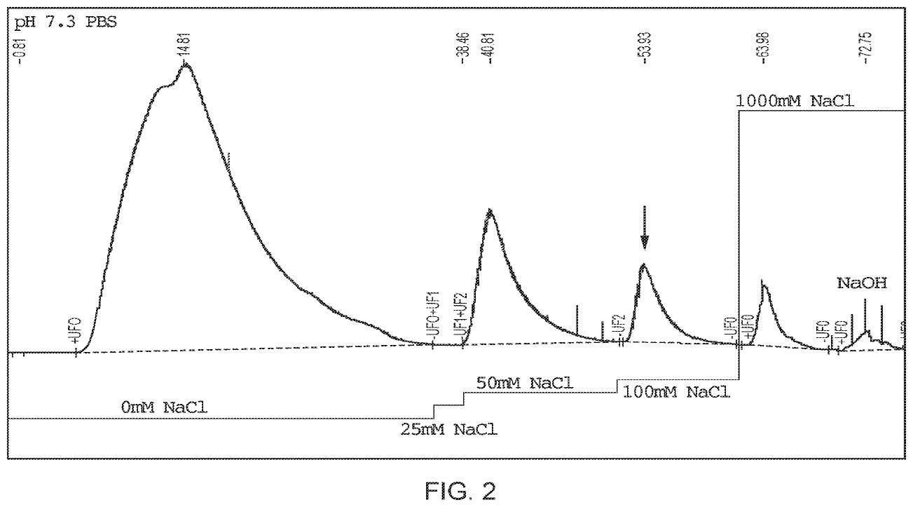

Table 3 provides a summary of the combined fractions and quantities of protein observed in each step (e.g., flow through, first wash, second wash, eluate, and cleaning). Furthermore, FIG. 2 provides a chromatogram of the purification.

TABLE 3
Purification using DETOXI-GEL ™, pH 7.3.

| | | | | IαIp ELISA (Heparin- | | IαIp | |
|---|---|---|---|---|---|---|---|
| | Vol. | Protein Assay (BCA) | | Sandwich) | | Recovery | |
| Fraction | (mL) | (μg/mL) | (μg) | (μg/mL) | (μg) | (%) | Purity (%) |
| Diluted | 10.0 | 12,172.382 | 121,723.820 | 72.797 | 727.970 | | 0.598 |
| human | | | | | | | |
| plasma | | | | | | | |
| Flow | 50.2 | 1,355.227 | 68,032.395 | 2.157 | 108.281 | 14.87 | 0.159 |
| through | | | | | | | |
| Wash 1 | 24.6 | 523.940 | 12,888.924 | 7.284 | 179.186 | 24.61 | 1.390 |
| (50 mM | | | | | | | |
| NaCl) | | | | | | | |
| Wash 2 | 16.7 | 283.641 | 4,736.806 | 14.093 | 235.353 | 32.33 | 4.969 |
| (100 mM | | | | | | | |
| NaCl) | | | | | | | |
| Eluate | 7.3 | 201.462 | 1,470.673 | 5.566 | 40.632 | 5.58 | 2.763 |
| (1,000 | | | | | | | |
| mM | | | | | | | |
| NaCl) | | | | | | | |
| Cleaning | 6.7 | — | — | 9.745 | 65.292 | 8.97 | — |
| (1.0M | | | | | | | |
| NaOH) | | | | | | | |
| | | Mass | 71.6% | Mass | 86.370% | | |
| | | Balance | | Balance | | | |

IαIps eluted in the second wash step, with 32.33% yield and 4.969% purity (for the chromatogram, please see FIG. 2A). Furthermore, IaαIps were detected in the flow through and in every wash fraction at pH 7.3. DETOXI-GEL™ is an endotoxin-binding agent that employs a polymyxin B resin as the endotoxin-binding agent. While this method is unoptimized, the next example demonstrates that at a different pH, DETOXI-GEL™ binds to IαIps with improved affinity. Furthermore, this example and the subsequent example demonstrate that DETOXI-GEL™ and other similar endotoxin-binding agents can selectively bind IaαIps, which was unexpected.

### Example 3. Purification of an IαIp from Human Plasma Using DETOXI-GEL™, pH 5.5

A sample of human plasma (fresh frozen plasma, 2.5 mL) was diluted (1:3 (v/v) dilution in 15 mM phosphate, pH 5.5) and the diluted plasma (10.0 mL) was applied to a 4 mL endotoxin-binding agent column (DETOXI-GEL™, Pierce) at a flow rate of 2 mL per minute. The flow through was collected for analysis and was then discarded. Additional dilution buffer (63.6 mL: 15 mM phosphate, pH 5.5) was applied to the column to allow the starting material to pass through the column completely. The additional flow through was collected for analysis and was discarded because no (or an insubstantial amount of) IαIp was detected.

The column was washed with a first wash buffer (26.4 mL: 15 mM phosphate+50 mM NaCl, pH 5.5) and the resulting fractions were collected, combined, and analyzed for total protein (BCA assay) and IαIp (MAb 69.26—heparin-biotin sandwich ELISA). The combined fractions were discarded because no (or an insubstantial amount of) IαIp was detected.

After the 50 mM NaCl first wash, the column was further washed with a second wash buffer having a higher concentration of NaCl (25.6 mL: 15 mM phosphate+100 mM NaCl, pH 5.5) and the resulting fractions were collected, combined, and analyzed (see Table 4). The combined fractions were determined to contain IαIp (3.691% pure, 281.958 μg, 38.73% yield of IαIp (w/w)).

After the 100 mM NaCl second wash, an elution buffer having a higher concentration of NaCl (14.5 mL: 15 mM phosphate+1,000 mM NaCl, pH 5.5) was applied to the endotoxin-binding agent and the resulting fractions were collected, combined and analyzed for IαIp. The combined eluate fractions were determined to contain a majority IαIp (20.578% pure, 566.979 μg, 77.88% yield of IαIp (w/w)) as shown in Table 4.

The column was further cleaned by washing with a high pH buffer (6.8 mL: 1 M NaOH) and the resulting fractions were collected, combined, and analyzed for IαIp. These combined fractions were determined to contain IαIp (43.255 μg, 5.94% yield of IαIp (w/w)) as shown in Table 4.

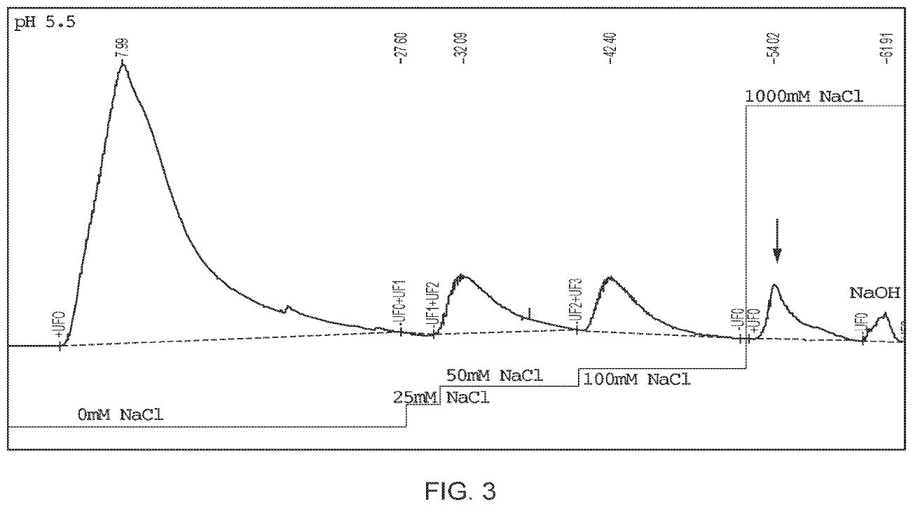

Table 4 provides a summary of the combined fractions and quantities of protein observed in each step (e.g., flow through, first wash, second wash, eluate, and cleaning). Furthermore, FIG. 3 provides a chromatogram of the purification.

TABLE 4
Purification using DETOXI-GEL ™, pH 5.5.

| | | | | IαIp ELISA (Heparin- | | IαIp | |
|---|---|---|---|---|---|---|---|
| | Vol. | Protein Assay (BCA) | | Sandwich) | | Recovery | |
| Fraction | (mL) | (μg/mL) | (μg) | (μg/mL) | (μg) | (%) | Purity (%) |
| Diluted | 10.0 | 12,172.382 | 121,723.820 | 72.797 | 727.970 | | 0.598 |
| human | | | | | | | |
| plasma | | | | | | | |
| Flow | 63.6 | 1,045.157 | 66,471.985 | 0.000 | 0.000 | 0.000 | 0.000 |
| through | | | | | | | |
| Wash 1 | 26.4 | 451.008 | 11,906.611 | 0.000 | 0.000 | 0.000 | 0.000 |
| (50 mM | | | | | | | |
| NaCl) | | | | | | | |
| Wash 2 | 25.6 | 298.441 | 7,640.090 | 11.014 | 281.958 | 38.73 | 3.691 |
| (100 mM | | | | | | | |
| NaCl) | | | | | | | |
| Eluate | 14.5 | 190.020 | 2,755.290 | 39.102 | 566.979 | 77.88 | 20.578 |
| (1,000 | | | | | | | |
| mM | | | | | | | |
| NaCl) | | | | | | | |
| Cleaning | 6.8 | — | — | 6.361 | 43.255 | 5.94 | — |
| (1.0M | | | | | | | |
| NaOH) | | | | | | | |
| | | Mass | 72.9% | Mass | 122.56% | | |
| | | Balance | | Balance | | | |

The majority of the IαIps eluted in the third wash step (1,000 mM NaCl), with 77.88% recovery of IαIp and 20.578% purity (for the chromatogram, please see FIG. 2B). Furthermore, at pH 5.5, IαIps were not detected in the flow through, nor the first wash fractions, demonstrating that DETOXI-GEL™ binds to IαIp selectively. This example confirms that endotoxin-binding agents can be used to purify IαIps from a biological material.

### Example 4. Purification of an IαIp from Human Plasma Using DETOXI-GEL™, pH 7.2

An eluate from a Q anion-exchange column (from fresh frozen human plasma) containing enriched IαIp (ca. 40% w/w) was applied (30 mL) to a 4 mL endotoxin-binding agent column (DETOXI-GEL™, Pierce) at a flow rate of 2 mL per minute. The flow through was collected for analysis for IαIp (MAb 69.26-heparin-biotin sandwich ELISA) and was discarded because no IαIp was detected.

The column was washed with a first wash buffer (14.6 mL: 20 mM Tris-HCl+150 mM NaCl, pH 7.2) and the resulting fractions were collected, combined, and analyzed for IαIp. It was determined that the combined fractions contained the majority of IαIp from the starting material (653.5982 μg, 75.3% recovery of IαIp (w/w)).

After the 150 mM NaCl wash, the column was further washed with a second wash buffer having a higher concentration of NaCl (5.8 mL: 20 mM Tris-HCl+250 mM NaCl, pH 7.2) and the resulting fractions were collected, combined, and analyzed (see Table 5, Wash 1). The combined wash fractions were determined to contain a smaller amount of IαIp from the previous wash step (44.1728 μg, 5.09% recovery of IαIp (w/w)).

After the 250 mM NaCl wash, the endotoxin-binding agent was further washed with an elution buffer having a higher concentration of NaCl (6.8 mL, 20 mM Tris-HCl+500 mM NaCl, pH 7.2). The resulting fractions were collected, combined, and analyzed for IαIp. These combined fractions contained more IαIp than the second wash step fractions (143.7316 μg, 16.56% recovery of IαIp (w/w)).

The column was further washed with another elution buffer having a higher concentration of NaCl (3.7 mL, 20 mM Tris-HCl+1,000 mM NaCl) and the resulting fractions were combined and analyzed for IαIp. These combined fractions contained less IαIp than the first eluate fractions (42.14 μg, 4.86% recovery of IαIp (w/w)).

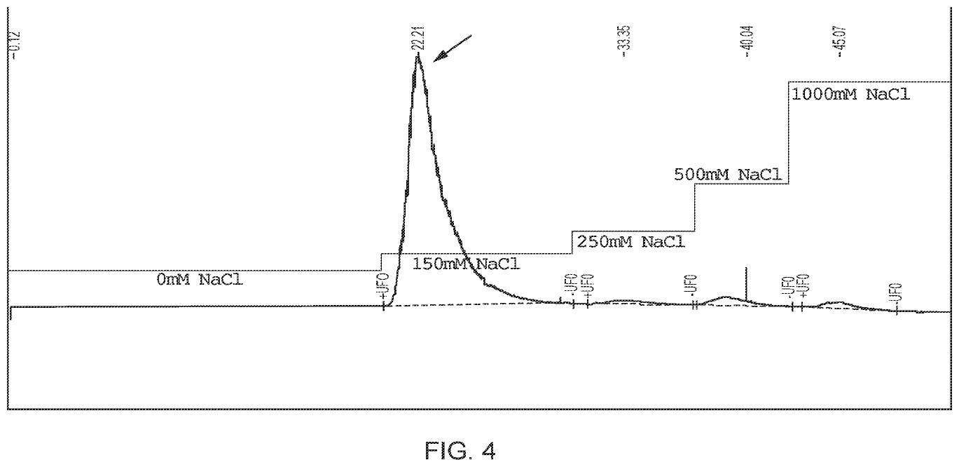

Table 5 provides a summary of the combined fractions and quantities of protein observed in each step (e.g., flow through, first wash, second wash, first eluate, and second eluate). Furthermore, FIG. 4 provides a chromatogram of the purification.

TABLE 5
Purification using DETOXI-GEL ™, pH 7.2.

| | | |
|---|---|---|
| | IαIp ELISA | IαIp |
| Vol. | (Heparin-Sandwich) | Recovery |

| Fraction | (mL) | (μg/mL) | (μg) | (%) |
|---|---|---|---|---|
| Q eluate of | 30 | 28.934 | 868.02 | — |
| human plasma | | | | |
| Flow through | 30.5 | 0.000 | 0.000 | 0.00 |
| Wash 1 (150 | 14.6 | 44.767 | 653.5982 | 75.3 |
| mM NaCl) | | | | |
| Wash 2 (250 | 5.8 | 7.616 | 44.1728 | 5.09 |
| mM NaCl) | | | | |
| Eluate 1 (500 | 6.8 | 21.137 | 143.7316 | 16.56 |
| mM NaCl) | | | | |
| Eluate 2 (1,000 | 3.7 | 11.39 | 42.14 | 4.86 |
| mM NaCl) | | | | |

Mass balance: 101.80%

The majority of the IaαIps eluted in the first wash step, along with most of the remaining proteins from the Q eluate (75.30% recovery of IαIp). Furthermore, IaαIps were detected in the other wash fraction and the two eluate fractions.

### Example 5. Purification of an IαIp from Cryo-Poor Plasma Using ETOXICLEAR™

A sample of cryo-poor plasma (11.5 mL) was diluted (1:4 (v/v): in 20 mM Tris-HCl pH 7.2+200 mM NaCl) and the diluted cryo-poor plasma was applied to a commercially available 5 mL Q anion-exchange resin (Tosoh TOYOPEARL® GigaCap Q650M) at a flow rate of 5 mL per minute (120 cm/hr). The flow through was collected for analysis. The flow through, which did not contain (or contained an insubstantial amount of) IαIp was discarded. Additional plasma dilution buffer (20 mM Tris-HCl pH 7.2+200 mM NaCl) was applied to the column to allow the starting material to pass through the column completely. The additional flow through was collected for analysis and was determined to contain no (or an insubstantial amount of) IαIp and was discarded.

The column was washed with a first wash buffer (20 mM Tris-HCl pH 7.2+250 mM NaCl) and the resulting fractions were collected. The combined fractions were analyzed for total protein (bicinchoninic acid assay (BCA)), IαIp (MAb 69.26—heparin-biotin sandwich ELISA), and trypsin inhibition activity as shown in Table 6. The combined wash fractions contained a small amount of impure IαIp and were discarded.

After the wash at pH 7.2, the column was further washed with a second wash buffer with a lower pH (50 mM Gly 100 mM AcOH 150 mM NaCl pH 5.2) and the resulting fractions were collected, combined, and analyzed as shown in Table 6. The combined wash fractions contained a small amount of impure IαIp and were discarded.

After the wash at pH 5.2, the bound protein was eluted with a high salt elution buffer (20 mM Tris-HCl pH 7.2+750 mM NaCl). The fractions were collected, combined, and analyzed (e.g., total protein, IαIp, and trypsin inhibition activity) as shown in Table 6. The collected fractions contained enriched IαIp.

A cleaning buffer with a high pH/high salt content (1 M NaOH+2 M NaCl) was next applied to the column and the resulting fractions were combined and analyzed for IαIp as described in Table 6. The combined fractions were determined to contain no (or an insubstantial amount of) IαIp and were discarded.

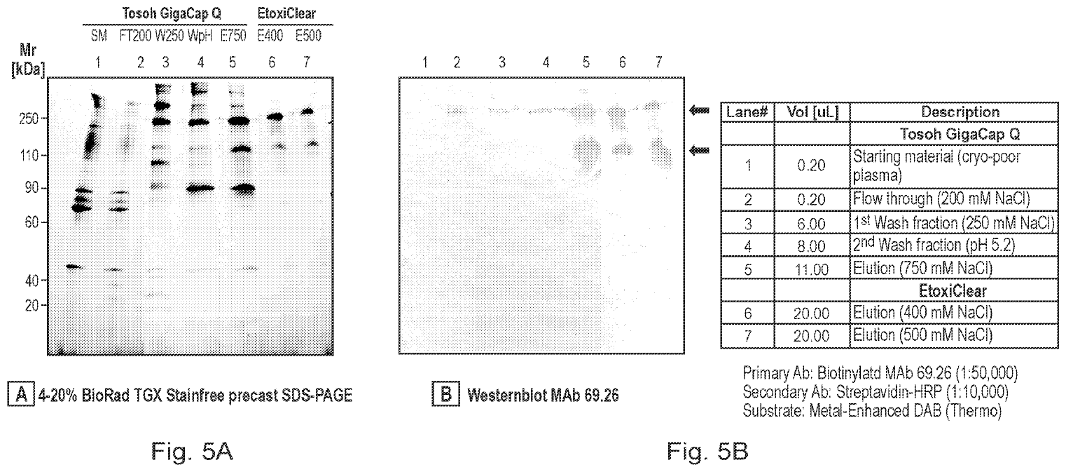

Table 6 provides a summary of the combined fractions and quantities of protein observed in each step (e.g., flow through, first wash, second wash, elution, and cleaning). Furthermore, FIG. 5A-5B show an SDS-Page gel and a Western blot, respectively, containing combined fractions from each step collected from the Tosoh TOYOPEARL® GigaCap Q650M column.

TABLE 6
Capture step using Q anion-exchange resin.

| | | | | IαIp ELISA | | | | | |
|---|---|---|---|---|---|---|---|---|---|
| | | | | (Heparin- | | Biological Activity | | | |
| | Vol. | Protein Assay (BCA) | | Sandwich) | | Trypsin Inhibition Activity | | | Purity |
| Fraction | (mL) | (μg/mL) | (μg) | (μg/mL) | (μg) | (IU/mL) | (IU) | (IU/mg) | (%) |
| Starting | 45.6 | 15466.979 | 705294.242 | 119.651 | 5456.063 | 638.850 | 29131.560 | 41.304 | 0.77% |
| Material | | | | | | | | | |
| Flow | 110.0 | 6037.915 | 664170.650 | 0 | 0 | n/d | n/d | n/d | 0% |
| through | | | | | | | | | |
| Wash 1 | 38.2 | 480.22 | 18343.716 | 4.676 | 178.623 | 89.062 | 3402.168 | 185.468 | 0.97% |
| (pH 7.2) | | | | | | | | | |
| Wash 2 | 22.2 | 306.345 | 6800.859 | 15.799 | 350.738 | n/d | n/d | n/d | 5.15% |
| (pH 5.2) | | | | | | | | | |
| Eluate | 19.8 | 477.329 | 9451.114 | 259.038 | 5128.952 | 323.840 | 6412.032 | 678.442 | 54.2% |
| Cleaning | 10.5 | n/d | — | 0.069 | 0.725 | n/d | n/d | n/d | — |
| | | Mass | 99.07% | Mass | 103.72% | Mass | | | |
| | | Balance | | Balance | | Balance | | | |

Step Yield: 94%

The eluate from the Q anion-exchange column containing IαIp was then diluted (1:5 (v/v)) in dH₂O. This fraction was applied to a commercially available 8 mL column containing an endotoxin-binding agent (ETOXICLEAR™, Astrea Bioseparations) at a flow rate of 3.5 mL per minute (120 cm/hr). The flow through was collected for analysis and then discarded because no (or an insubstantial amount of) IαIp was detected. A flow through buffer 20 mM Tris-HCl pH 7.2+200 mM NaCl) was applied to the column. The flow through was collected for analysis and then discarded (no (or an insubstantial amount of) IαIp was detected).

The column was washed with a first wash buffer (50 mM Gly 100 mM AcOH+200 mM NaCl pH 5.2) and the resulting fractions were collected. The fractions were combined and analyzed for total protein (bicinchoninic acid assay (BCA)), IαIp (69.26 Mab−heparin-biotin sandwich ELISA), and trypsin inhibition activity as shown in Table 7. The combined wash fractions were then discarded (no (or an insubstantial amount of) IαIp was detected).

The column was further washed with a second wash buffer having a higher pH (20 mM Tris-HCl pH 7.2+300 mM NaCl) and the resulting fractions were collected and combined. The combined wash fractions contained a small amount of impure IαIp and were discarded.

The bound protein was then eluted from the column by applying a first high salt elution buffer (20 mM Tris-HCl pH 7.2+400 mM NaCl). The bound protein was then eluted from the column by applying a second high salt elution buffer (20 mM Tris-HCl pH 7.2+500 mM NaCl). The bound protein was then eluted from the column by applying a third high salt elution buffer (20 mM Tris-HCl pH 7.2+1000 mM NaCl). The resulting fractions were collected, combined, and analyzed as shown in Table 7.

The column was cleaned by applying a cleaning buffer (1 M NaOH, 2M NaCl); no additional (or an insubstantial amount of) IαIp was detected in the flow through.

Table 7 provides a summary of the combined fractions and quantities of protein observed in each step (e.g., flow through, first wash, second wash, eluate, second eluate, and cleaning). Furthermore, FIG. 5A-5B show the results of testing by SDS-PAGE and Western blot of combined fractions for each step collected from the ETOXICLEAR™ column.

TABLE 7
Purification using ETOXICLEAR ™

| | | | | IαIp ELISA | | | | | |
|---|---|---|---|---|---|---|---|---|---|
| | | Protein Assay | | (Heparin- | | Biological Activity | | | |
| | Vol. | (BCA) | | Sandwich) | | Trypsin Inhibition Activity | | | Purity |
| Fraction | (mL) | (μg/mL) | (μg) | (μg/mL) | (μg) | (IU/mL) | (IU) | (IU/mg) | (%) |
| Starting | 100.0 | 94.511 | 9451.114 | 51.290 | 5128.952 | 64.120 | 6412.032 | 678.442 | 54.2% |
| Material | | | | | | | | | |
| Flow | 113.3 | — | — | 0.027 | 3.059 | 0.317 | 35.916 | | |
| through + | | | | | | | | | |
| Wash 1 | | | | | | | | | |
| (pH 5.2) | | | | | | | | | |
| Wash 2 | n/d | n/d | n/d | n/d | n/d | n/d | n/d | n/d | |
| (300 mM | | | | | | | | | |
| NaCl) | | | | | | | | | |
| Eluate 1 | 26.3 | 185.658 | 4882.805 | 183.849 | 4835.216 | 187.812 | 4939.456 | 1011.602 | 99.02% |
| (400 mM | | | | | | | | | |
| NaCl) | | | | | | | | | |
| Eluate 2 | 14.3 | 45.512 | 650.822 | 17.130 | 244.959 | 57.450 | 821.535 | 1262.304 | 37% |
| (500 mM | | | | | | | | | |
| NaCl) | | | | | | | | | |
| Eluate 3 | 17.2 | 46.044 | 791.957 | 1.429 | 24.579 | 30.311 | 521.349 | 658.305 | 3.1% |
| (1000 | | | | | | | | | |
| mM | | | | | | | | | |
| NaCl) | | | | | | | | | |
| Cleaning | 12.1 | 38.895 | 470.630 | 0.140 | 1.694 | n/d | n/d | n/d | |
| | | Mass | 71.91% | Mass | 99.62% | Mass | 98.54% | | |
| | | Balance | | Balance | | Balance | | | |

Step Yield: 94.27%
Overall Yield: 88.6%

### Example 6. Purification of an IαIp from Cryo-Poor Plasma Using ETOXICLEAR™

A sample of cryo-poor plasma (50 mL) was diluted (1:4 (v/v): in 20 mM Tris-HCl pH 7.2+200 mM NaCl) and the diluted cryo-poor plasma was applied to a commercially available 100 mL Q anion-exchange resin (Tosoh TOYOPEARL® GigaCap Q650M) at a flow rate of 12 mL per minute (120 cm/hr). The flow through was collected for analysis. The flow through, which did not contain (or contained an insubstantial amount of) IαIp was discarded. Additional plasma dilution buffer (20 mM Tris-HCl pH 7.2+200 mM NaCl) was applied to the column to allow the starting material to pass through the column completely. The additional flow through was collected for analysis and was determined to contain no (or an insubstantial amount of) IαIp and was discarded.

The column was washed with a first wash buffer (20 mM Tris-HCl pH 7.2+250 mM NaCl) and the resulting fractions were collected. The combined fractions were analyzed for total protein (bicinchoninic acid assay (BCA)), IαIp (MAb 69.26—heparin-biotin sandwich ELISA), and trypsin inhibition activity as shown in Table 8. The combined wash fractions contained a small amount of impure IαIp and were discarded.

After the wash at pH 7.2, the column was further washed with a second wash buffer with a lower pH (50 mM Gly 100 mM AcOH 150 mM NaCl pH 5.2) and the resulting fractions were collected, combined, and analyzed as shown in Table 8. The combined wash fractions contained a small amount of impure IαIp and were discarded.

After the wash at 5.2, the bound protein was eluted with a high salt elution buffer (20 mM Tris-HCl pH 7.2+750 mM NaCl). The fractions were collected, combined, and analyzed (e.g., total protein, IαIp, and trypsin inhibition activity) as shown in Table 8. The collected fractions contained enriched IαIp.

A cleaning buffer with a high pH/high salt content (1 M NaOH+2 M NaCl) was next applied to the column and the resulting fractions were combined and analyzed for IαIp as described in Table 8. The combined fractions were determined to contain no (or an insubstantial amount of) IαIp and were discarded.

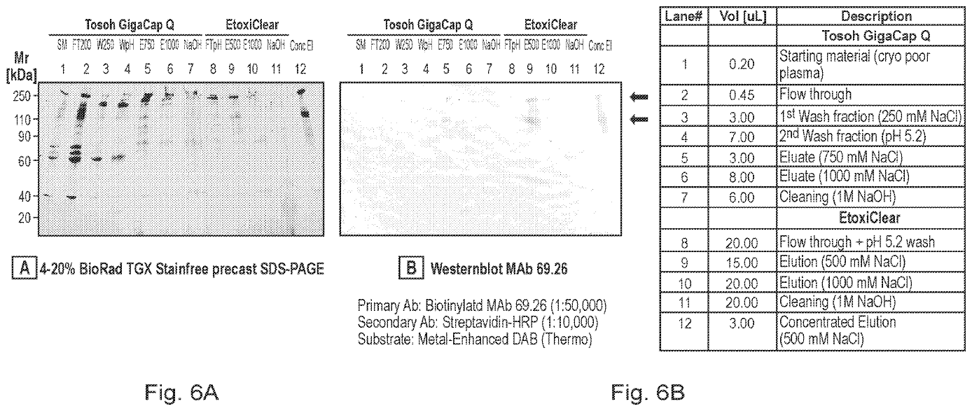

Table 8 provides a summary of the combined fractions and quantities of protein observed in each step (e.g., flow through, first wash, second wash, elution, and cleaning). Furthermore, FIG. 6A-6B show an SDS-Page gel and a Western blot, respectively, containing combined fractions from each step collected from the Tosoh TOYOPEARL® GigaCap Q650M column.

TABLE 8
Capture step using Q anion-exchange resin.

| | | | | IαIp ELISA | | | | | |
|---|---|---|---|---|---|---|---|---|---|
| | | | | (Heparin- | | Biological Activity | | | |
| | Vol. | Protein Assay (BCA) | | Sandwich) | | Trypsin Inhibition Activity | | | Purity |
| Fraction | (mL) | (μg/mL) | (μg) | (μg/mL) | (μg) | (IU/mL) | (IU) | (IU/mg) | (%) |
| Starting | 200 | 18606.907 | 3721381.400 | 105.477 | 21095.300 | 5414.742 | 1082948.40 | 291.007 | 0.56% |
| Material | | | | | | | | | |
| Flow | 481.2 | 6964.328 | 3351234.634 | n/d | n/d | 1979.453 | 952512.784 | 284.227 | |
| through | | | | | | | | | |
| Wash 1 | 117.3 | 928.419 | 108903.549 | 0.816 | 95.717 | 448.775 | 52641.308 | 483.376 | 0.08% |
| (pH 7.2) | | | | | | | | | |
| Wash 2 | 141.6 | 476.973 | 67539.377 | 0.545 | 77.172 | n/d | n/d | n/d | 0.114% |
| (pH 5.2) | | | | | | | | | |
| Eluate 1 | 126.3 | 1196.612 | 151132.096 | 161.332 | 20376.232 | 591.013 | 74644.942 | 493.905 | 13.48% |
| (750 | | | | | | | | | |
| mM) | | | | | | | | | |
| Eluate 1 | 28.2 | 385.681 | 10876.204 | 25.627 | 722.681 | n/d | n/d | n/d | 6.6% |
| (1000 | | | | | | | | | |
| mM) | | | | | | | | | |
| Cleaning | 59.6 | 503.206 | 29991.078 | n/d | n/d | n/d | n/d | n/d | — |
| | | Mass | 99.95% | Mass | 100.84% | Mass | 99.71% | | |
| | | Balance | | Balance | | Balance | | | |

Step Yield: 96%

The eluate from the Q anion-exchange column containing IαIp was then diluted (1:5 (v/v)) in dH₂O to 125 mL. This fraction was applied to a commercially available 74 mL column containing an endotoxin-binding agent (ETOXICLEAR™, Astrea Bioseparations) at a flow rate of 3.5 mL per minute (120 cm/hr). The flow through was collected for analysis and then discarded because no (or an insubstantial amount of) IαIp was detected. A flow through buffer 20 mM Tris-HCl pH 7.2+200 mM NaCl) was applied to the column. The flow through was collected for analysis and then discarded (no (or an insubstantial amount of) IαIp was detected).

The column was washed with a first wash buffer (50 mM Gly 100 mM AcOH+200 mM NaCl pH 5.2) and the resulting fractions were collected. The fractions were combined and analyzed for total protein (bicinchoninic acid assay (BCA)), IαIp (69.26 Mab−heparin-biotin sandwich ELISA), and trypsin inhibition activity as shown in Table 9. The combined wash fractions were then discarded (no (or an insubstantial amount of) IαIp was detected).

The column was further washed with a second wash buffer having a higher pH (20 mM Tris-HCl pH 7.2+300 mM NaCl) and the resulting fractions were collected and combined. The combined wash fractions contained a small amount of impure IαIp and were discarded.

The bound protein was then eluted from the column by applying a first high salt elution buffer (20 mM Tris-HCl pH 7.2+500 mM NaCl). The bound protein was then eluted from the column by applying a second high salt elution buffer (20 mM Tris-HCl pH 7.2+1000 mM NaCl). The resulting fractions were collected, combined, and analyzed as shown in Table 9.

The column was cleaned by applying a cleaning buffer (1 M NaOH, 2M NaCl); no additional (or an insubstantial amount of) IαIp was detected in the flow through.

Table 9 provides a summary of the combined fractions and quantities of protein observed in each step (e.g., flow through, first wash, second wash, eluate, second eluate, and cleaning). Furthermore, FIG. 6A-6B show the results of testing by SDS-PAGE and Western blot of combined fractions for each step collected from the ETOXICLEAR™ column.

TABLE 9
Purification using ETOXICLEAR ™

| | | | | IαIp ELISA | | | | | |
|---|---|---|---|---|---|---|---|---|---|
| | | Protein Assay | | (Heparin- | | Biological Activity | | | |
| | Vol. | (BCA) | | Sandwich) | | Trypsin Inhibition Activity | | | Purity |
| Fraction | (mL) | (μg/mL) | (μg) | (μg/mL) | (μg) | (IU/mL) | (IU) | (IU/mg) | (%) |
| Starting | 625.0 | 239.322 | 149576.500 | 32.266 | 20166.500 | 118.203 | 73876.625 | 493.90 | 13.48% |
| Material | | | | | | | | | |
| Flow | 778.5 | 125.926 | 98033.391 | 0.180 | 140.130 | n/d | n/d | n/d | 0.14% |
| through + | | | | | | | | | |
| Wash 1 | | | | | | | | | |
| (pH 5.2) | | | | | | | | | |
| Wash 2 | — | — | — | — | — | — | — | — | — |
| (300 mM | | | | | | | | | |
| NaCl) | | | | | | | | | |
| Eluate 1 | 115.4 | 196.871 | 22718.913 | 168.163 | 19406.049 | 282.1573256 | 0.918 | 1433.208 | 85.41% |
| (500 mM | | | | | | | | | |
| NaCl) | | | | | | | | | |
| Eluate 2 | 88.4 | 59.450 | 5255.380 | n/d | n/d | 80.039 | 7075.448 | 1346.325 | |
| (1000 | | | | | | | | | |
| mM | | | | | | | | | |
| NaCl) | | | | | | | | | |
| Cleaning | 61.8 | 44.808 | 2769.134 | n/d | n/d | n/d | n/d | n/d | |
| | | Mass | 86.09% | Mass | 96.92% | Mass | 56.23% | | |
| | | Balance | | Balance | | Balance | | | |

Step Yield: 96.23%
Overall Yield: 91.99%

### Example 7. Analysis of Copurified Impurity: Factor II (Prothrombin)

Factor II (Prothrombin) copurified with IαIp in Example 5-6 was analyzed by ELISA (Table 10). Sandwich ELISA were performed in a 96 well-plate (NUNC Immuno MAXISORP F96) with paired commercially available polyclonal antibodies applying a peroxidase-labelled detection antibody and reaction detection by relative absorbance signal at 450 nm. ELISA plates were coated overnight with a polyclonal sheep anti-human prothrombin (1:1000 in coat buffer), washed 3 times with wash buffer, blocked with 0.1% Milch, 2 mmol/L Benzamidine in wash buffer (block buffer), and washed with wash buffer. Test material and standard calibration samples (both at a chosen dilution range with block buffer) were incubated on the ELISA coated plates during 1 h at room temperature. Standard calibration typically performed on a five-point calibration curve using a commercially available reference plasma preparation (CRYOcheck, PrecisionBioLogic), regularly checked against the secondary international standard ISTH/SSC with a certified FII activity.

Incubated ELISA plates were washed three times and incubated 1 h at room temperature with sheep anti human prothrombin-peroxidase (HRP)-labelled detection antibody. Washed 3 times with wash buffer, and the HRP chromogenic reaction was triggered by TMB reagent (3,3′,5,5′ tetramethylbenzidine dihydrochloride) and prothrombin level measured by the chromogenic detection signal at 450 nm

TABLE 10
ELISA Factor II (ProThrombin)

| | Cryo-poor plasma | | Cryo-poor plasma | |
|---|---|---|---|---|
| | (example 5) | | (example 6) | |
| | FII ELISA | FII reduction | FII ELISA | FII reduction |
| Fraction | [mU/mL] | (%) | [mU/mL] | (%) |
| Starting Material | 716 | — | 785 | — |
| of anion- | | | | |
| exchange | | | | |
| chromatography | | | | |
| Eluate 1 (500 | 4.58 | 99.4% | 2.9 | 99.6% |
| mM NaCl) of | | | | |
| endotoxin | | | | |
| binding agent | | | | |
| chromatography | | | | |

### OTHER EMBODIMENTS

All publications, patents, and patent applications mentioned in the above specification are hereby incorporated by reference to the same extent as if each individual publication, patent or patent application was specifically and individually indicated to be incorporated by reference in its entirety. Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method of purifying an inter-alpha inhibitor protein (IαIp) from a biological material comprising:
(a) applying the biological material comprising the IαIp to an endotoxin-binding agent and separating a flow through comprising the biological material that does not bind to the endotoxin-binding agent; and
(b) applying an elution buffer comprising a salt to the endotoxin-binding agent and collecting an eluate comprising the IαIp.

2. The method of claim 1, wherein the endotoxin-binding agent is immobilized on a support.

3. The method of claim 2, wherein the support is a monolithic support or a particle-based support.

4. The method of claim 3, wherein the monolithic support or particle-based support is or comprises a resin.

5. The method of any one of claims 2-4, wherein the support comprises a column, membrane, disc, or chip.

6. The method of any one of claims 1-5, wherein the endotoxin-binding agent is selected from the group consisting of ETOXICLEAR™, PIERCE™ High Capacity Endotoxin Removal Resin, TOXINERASER™ Endotoxin Removal Resin, PURKINE™ Endotoxin Removal Resin, DETOXI-GEL™ Endotoxin Removing Gel, and PROMEGA™ Endotoxin Removal Resin.

7. The method of claim 6, wherein the endotoxin-binding agent is DETOXI-GEL™ or ETOXICLEAR™.

8. The method of any one of claims 1-7, wherein the biological material further comprises three or more proteins selected from the group consisting of alpha-1 antitrypsin, C1-inhibitor, albumin, a globulin, fibrinogen (factor I), prothrombin (factor II), thrombin, anti-thrombin III, factor III, factor V, factor VII, factor VIII, factor IX, factor X, factor XI, factor XII, factor XIII, fibronectin, alpha-2 antiplasmin, urokinase, protein C, protein S, protein Z, protein Z-related protease inhibitor, plasminogen, tissue plasminogen activator, plasminogen activator inhibitor-1, plasminogen activator inhibitor-2, von Willebrand factor, factor H, prekallikrein, high-molecular-weight kininogen, and heparin cofactor II.

9. The method of claim 8, wherein the globulin is an immunoglobulin (Ig).

10. The method of claim 9, wherein the immunoglobulin is selected from the group consisting of IgA, IgE, IgM, IgD, and IgG.

11. The method of claim 10, wherein the IgG is selected from the group consisting of intravenous Ig (IVIg), anti-D IgG, hepatitis B IgG, measles IgG, rabies IgG, tetanus IgG, and Varicella Zoster IgG.

12. The method of any one of claims 1-11, wherein the biological material comprises three to ten three to fifteen, three to twenty, three to twenty five, three to thirty, ten to twenty, ten to twenty five, ten to thirty, fifteen to twenty five, fifteen to thirty, twenty to thirty, or thirty or more different proteins.

13. The method of any one of claims 1-12, wherein the biological material comprises about 40 to about 65% albumin (w/w).

14. The method of any one of claims 1-13, wherein the biological material comprises about 25 to about 45% globulins (w/w).

15. The method of any one of claims 1-14, wherein the biological material comprises about 2 to about 12% fibrinogen (w/w).

16. The method of any one of claims 1-15, wherein the method further comprises applying a first wash buffer to the endotoxin-binding agent after step (a) and prior to step (b).

17. The method of claim 16, wherein the method further comprises separating a flow through comprising the first wash buffer.

18. The method of claim 16 or 17, wherein the first wash buffer has a pH of about 4.5 to 8.5.

19. The method of claim 18, wherein the first wash buffer has a pH of about 5.2.

20. The method of any one of claims 16-19, wherein the first wash buffer comprises one or more of glycine, acetic acid, citric acid, phosphate, sodium chloride (NaCl), calcium, magnesium, EDTA, and Tris-HCl.

21. The method of any one of claims 16-20, wherein the first wash buffer comprises about 10 to about 200 mM glycine and/or about 20 to 300 mM acetic acid.

22. The method of claim 21, wherein the first wash buffer comprises about 75 mM glycine and about 100 mM acetic acid.

23. The method of any one of claims 16-22, wherein the first wash buffer comprises about 200 mM or less NaCl.

24. The method of claim 23, wherein the first wash buffer comprises about 50 to about 150 mM NaCl.

25. The method of claim 24, wherein the first wash buffer comprises about 50 mM NaCl or about 100 mM NaCl.

26. The method of any one of claims 16-25, wherein the method further comprises applying a second wash buffer to the endotoxin-binding agent after applying the first wash buffer.

27. The method of claim 26, wherein the method further comprises separating a flow through comprising the second wash buffer.

28. The method of claim 26 or 27, wherein the second wash buffer has a pH of about 4.5 to about 8.5.

29. The method of claim 28, wherein the second wash buffer has a pH of about 7.2.

30. The method of any one of claims 26-29, wherein the second wash buffer comprises one or more of glycine, acetic acid, citric acid, phosphate, sodium chloride (NaCl), calcium, magnesium, EDTA, and Tris-HCl.

31. The method of any one of claims 26-30, wherein the second wash buffer comprises about 5 to about 100 mM Tris-HCl.

32. The method of claim 31, wherein the second wash buffer comprises about 20 mM Tris-HCl.

33. The method of any one of claims 26-32, wherein the second wash buffer comprises about 500 mM NaCl or less.

34. The method of claim 33, wherein the second wash buffer comprises about 100 to about 500 mM NaCl.

35. The method of claim 34, wherein the second wash buffer comprises about 300 mM NaCl.

36. The method of any one of claims 1-35, wherein the elution buffer has a pH of about 4.5 to about 8.5.

37. The method of claim 36, wherein the elution buffer has a pH of about 7.2.

38. The method of any one of claims 1-37, wherein the elution buffer comprises one or more of glycine, acetic acid, citric acid, phosphate, sodium chloride (NaCl), calcium, magnesium, EDTA, and Tris-HCl.

39. The method of any one of claims 1-38, wherein the elution buffer comprises about 5 to about 100 mM Tris-HCl.

40. The method of claim 39, wherein the elution buffer comprises about 20 mM Tris-HCl.

41. The method of any one of claims 1-40, wherein the elution buffer comprises about 1,000 mM NaCl or less.

42. The method of any one of claims 1-41, wherein the elution buffer comprises about 500 to about 1,000 mM NaCl.

43. The method of claim 41 or 42, wherein the elution buffer comprises about 500 mM NaCl or about 1,000 mM NaCl.

44. The method of any one of claims 1-43, the method further comprises applying a dilution buffer to the biological material prior to step (a).

45. The method of claim 44, wherein the dilution buffer comprises deionized water.

46. The method of claim 44 or 45, wherein the dilution buffer has a pH of about 4.5 to about 8.5.

47. The method of claim 46, wherein the dilution buffer has a pH of about 5.5, about 7.2, or about 7.3.

48. The method of any one of claims 44-47, wherein the biological material is diluted 1:1 to 1:10 (v/v) with the dilution buffer.

49. The method of any one of claims 44-48, wherein the dilution buffer comprises one or more of glycine, acetic acid, citric acid, phosphate, sodium chloride (NaCl), calcium, magnesium, EDTA, and Tris-HCl.

50. The method of any one of claims 44-49, wherein the dilution buffer comprises about 5 to about 100 mM Tris-HCl.

51. The method of claim 50, wherein the dilution buffer comprises about 20 mM Tris-HCl.

52. The method of any one of claims 44-51, wherein the dilution buffer comprises about 50 mM NaCl or less.

53. The method of claim 52, wherein the dilution buffer comprises no salt or about 50 mM NaCl.

54. The method of any one of claims 44-53, wherein the dilution buffer comprises about 5 to about 100 mM phosphate.

55. The method of claim 54, wherein the dilution buffer comprises about 15 mM phosphate.

56. The method of any one of claims 1-55, wherein the method further comprises detecting an amount of the IαIp in the flow through.

57. The method of claim 56, wherein the method comprises discarding the flow through.

58. The method of any one of claims 1-57, wherein the method further comprises detecting an amount of IαIp in the eluate.

59. The method of any one of claims 1-58, the method comprises a flow rate of about 1 to 10 mL/minute.

60. The method of any one of claims 1-58, wherein, prior to step (a), the method comprises applying the biological material to a chromatography support.

61. The method of claim 60, wherein the chromatography support comprises an anion-exchange chromatography support, a size-exclusion chromatography support, an ion-exchange chromatography support, an affinity chromatography support, or a combination thereof.

62. The method of claim 61, wherein the chromatography support is said anion-exchange chromatography support.

63. The method of any one of claims 60-62, wherein the method further comprises:
(i) applying the biological material to the chromatography support and separating a flow through of step (i) comprising the biological material that does not bind to the chromatography support; and
(ii) applying an elution buffer comprising a salt to the chromatography support and collecting a first eluate comprising the IαIp.

64. The method of claim 63, wherein the chromatography support is a monolithic support or a particle-based support.

65. The method of claim 64, wherein the monolithic support or particle-based support comprises an immobilized anion-exchange resin.

66. The method of claim 65, wherein the immobilized anion-exchange resin is diethylaminoethane (DEAE) resin or a quaternary amine (Q) resin.

67. The method of any one of claims 60-66, wherein the chromatography support is a column, membrane, disc, or chip.

68. The method of any one of claims 60-67, wherein the method further comprises applying a first wash buffer to the chromatography support after step (i) and prior to step (ii).

69. The method of claim 68, wherein, prior to step (ii), the method further comprises separating a flow through comprising the first wash buffer.

70. The method of claim 68 or 69, wherein the first wash buffer applied to the chromatography support has a pH of about 4.5 to about 8.5.

71. The method of claim 70, wherein the first wash buffer applied to the chromatography support has a pH of about 7.2.

72. The method of claim any one of claims 68-71, wherein the first wash buffer applied to the chromatography support comprises one or more of glycine, acetic acid, citric acid, phosphate, sodium chloride (NaCl), calcium, magnesium, EDTA, and Tris-HCl.

73. The method of any one of claims 68-72, wherein the first wash buffer applied to the chromatography support comprises about 5 to about 100 mM Tris-HCl.

74. The method of claim 73, wherein the first wash buffer applied to the chromatography support comprises about 20 mM Tris-HCl.

75. The method of any one of claims 68-74, wherein the first wash buffer applied to the chromatography support comprises about 400 mM or less NaCl.

76. The method of claim 75, wherein the first wash buffer applied to the chromatography support comprises about 50 to about 250 mM NaCl.

77. The method of claim 76, wherein the first wash buffer applied to the chromatography support comprises about 250 mM NaCl.

78. The method of any one of claims 68-77, wherein the method further comprises applying a second wash buffer to the chromatography support after applying the first wash buffer.

79. The method of claim 78, wherein the method further comprises separating a flow through comprising the second wash buffer.

80. The method of claim 78 or 79, wherein the second wash buffer applied to the chromatography support has a pH of about 4.5 to about 8.5.

81. The method of claim 80, wherein the second wash buffer applied to the chromatography support has a pH of about 5.2.

82. The method of any one of claims 78-81, wherein the second wash buffer applied to the chromatography support comprises one or more of glycine, acetic acid, citric acid, phosphate, sodium chloride (NaCl), calcium, magnesium, EDTA, and Tris-HCl.

83. The method of any one of claims 78-82, wherein the second wash buffer applied to the chromatography support comprises about 10 to about 200 mM glycine and/or about 20 to about 300 mM acetic acid.

84. The method of claim 83, wherein the second wash buffer applied to the chromatography support comprises about 50 mM glycine and about 100 mM acetic acid.

85. The method of any one of claims 78-84, wherein the second wash buffer applied to the chromatography support comprises about 100 to about 500 mM NaCl.

86. The method of claim 85, wherein the second wash buffer applied to the chromatography support comprises about 175 mM NaCl.

87. The method of any one of claims 63-86, wherein the elution buffer applied to the chromatography support has a pH of about 4.5 to about 8.5.

88. The method of claim 87, wherein the elution buffer applied to the chromatography support has a pH of about 7.2.

89. The method of any one of claims 63-88, wherein the elution buffer applied to the chromatography support comprises one or more of glycine, acetic acid, citric acid, phosphate, sodium chloride (NaCl), calcium, magnesium, EDTA, and Tris-HCl.

90. The method of any one of claims 63-89, wherein the elution buffer applied to the chromatography support comprises about 5 to about 100 mM Tris-HCl.

91. The method of claim 90, wherein the elution buffer applied to the chromatography support comprises about 20 mM Tris-HCl.

92. The method of any one of claims 63-91, wherein the elution buffer applied to the chromatography support comprises about 1,000 or less mM NaCl.

93. The method of claim 92, wherein the elution buffer applied to the chromatography support comprises about 750 mM NaCl.

94. The method of any one of claims 63-93, the method further comprises applying a dilution buffer to the biological material prior to step (i).

95. The method of claim 94, wherein the dilution buffer comprises deionized water.

96. The method of claim 94 or 95, wherein the dilution buffer has a pH of about 4.5 to about 8.5.

97. The method of claim 96, wherein the dilution buffer has a pH of about 7.2.

98. The method of any one of claims 94-97, wherein the biological material is diluted 1:1 to 1:10 (v/v) with the dilution buffer.

99. The method of any one of claims 94-98, wherein the dilution buffer comprises one or more of glycine, acetic acid, citric acid, phosphate, sodium chloride (NaCl), calcium, magnesium, EDTA, and Tris-HCl.

100. The method of any one of claims 94-99, wherein the dilution buffer comprises about 5 to about 100 mM Tris-HCl.

101. The method of claim 100, wherein the dilution buffer comprises about 20 mM Tris-HCl.

102. The method of any one of claims 94-101, wherein the dilution buffer comprises about 300 mM NaCl or less.

103. The method of claim 102, wherein the dilution buffer comprises no salt or about 200 mM NaCl.

104. The method of any one of claims 63-103, wherein the method further comprises detecting an amount of IαIp in the flow through of step (i).

105. The method of claim 104, wherein the method comprises discarding the flow through of step (i).

106. The method of any one of claims 63-105, the method further comprises detecting an amount of IαIp in the eluate in step (ii).

107. The method of any one of claims 63-106, the method comprises a flow rate of about 1 to 10 mL/minute.

108. The method of any one of claims 1-107, wherein the IαIp collected in the eluate of step (b) has a purity of about 5% to 99% or greater by weight relative to the purity of the IαIp in the biological material.

109. The method of any one of claims 1-108, wherein the yield of IαIp in the eluate collected in step (b) is greater than about 20% (w/w) relative to the IαIp present in the biological material.

110. The method of claim 109, wherein the yield is about 35% to about 90% or greater (w/w) relative to the IαIp present in the biological material.

111. The method of claim 110, wherein the yield IαIp is about 95% or greater by (w/w) relative to the IαIp present in the biological material.

112. The method of any one of claims 109-111, wherein the yield of IαIp from the biological material is at least about 5 μg/ml.

113. The method of claim 112, wherein the yield of IαIp from the biological material is at least about 50 μg/ml.

114. The method of claim 113, wherein the yield of IαIp from the biological material is at least about 100 μg/ml.

115. The method of claim 114, wherein the yield of IαIp from the biological material is at least about 300 μg/ml.

116. The method of claim 115, wherein the yield of IαIp from the biological material is at least about 600 μg/ml.

117. The method of claim 116, wherein the yield of IαIp from the biological material is at least about 900 μg/ml.

118. The method of any one of claims 108-117, wherein the purity of the IαIp is at least about 5% (w/w).

119. The method of claim 118, wherein the purity of the IαIp is at least about 25% (w/w).

120. The method of claim 119, wherein the purity of the IαIp is at least about 50% (w/w).

121. The method of claim 120, wherein the purity of the IαIp is at least about 75% (w/w).

122. The method of any one of claims 1-121, wherein the IαIp comprises two or more of inter-alpha inhibitor (IαI), pre-alpha inhibitor (PαI), and bikunin.

123. The method of any one of claims 1-122, wherein the IαIp present in the biological material comprises between 60% to 80% (w/w) IαIp and/or between 20% to 40% (w/w) PαI; and/or wherein the IαIp present in the eluate of step (b) comprises between 60% to 80% (w/w) IαIp and/or between 20% to 40% (w/w) PαI.

124. The method of any one of claims 1-123, wherein the IαIp has an apparent molecular weight of between about 60 to about 280 kDa.

125. The method of any one of claims 1-124, wherein the IαIp has biological activity.

126. The method of claim 125, wherein the biological activity comprises cytokine inhibitor activity, chemokine inhibitor activity, or serine protease inhibitor activity.

127. The method of any one of claims 1-126, wherein the biological material is a blood product material.

128. The method of claim 127, wherein the blood product material is selected from the group consisting of whole plasma, cryo-poor plasma, liquid plasma, frozen plasma (FP), source plasma, recovered plasma, solvent/detergent-treated plasma (SDP), platelet-rich plasma (PRP), platelet-poor plasma (PPP), serum, whole blood, and a diluted or concentrated preparation thereof.

129. The method of claim 128, wherein the FP is selected from the group consisting of fresh frozen plasma (FFP), FFP24, FP24, thawed FFP, thawed FFP24, thawed FP, thawed FP24, and a diluted or concentrated preparation thereof.

130. The method of any one of claims 1-126, wherein the biological material is milk or colostrum.

131. The method of any one of claims 1-130, wherein the biological material is from a mammal.

132. The method of claim 131, wherein the mammal is a human, primate, bovine, equine, porcine, ovine, feline, or canine.

133. The method of any one of claims 1-59, wherein the biological material is substantially unprocessed prior to application to the endotoxin-binding agent.

134. The method of any one of claims 1-133, wherein the method further comprises performing one or more chromatography steps using the eluate collected in step (b).

135. The method of claim 134, wherein the one or more additional chromatography steps comprises repeating the method of any one of claims 1-134.

136. The method of any one of claims 1-135, wherein the elution buffer applied to the endotoxin-binding agent in step (b) has a pH of 7.2 and comprises about 20 mM Tris-HCl and about 500 mM NaCl.

137. The method of any one of claims 16-35 wherein the first wash buffer applied to the endotoxin-binding agent has a pH of 5.2 and comprises about 75 mM glycine, about 100 mM acetic acid, and about 150 mM NaCl.

138. The method of any one of claims 26-35, wherein the second wash buffer applied to the endotoxin-binding agent has a pH of 7.2 and comprises about 20 mM Tris-HCl and about 300 mM NaCl.

139. The method of any one of claims 63-107, wherein the elution buffer applied to the chromatography support has a pH of 7.2 and comprises about 20 mM Tris-HCl and about 750 mM NaCl.

140. The method of any one of claims 68-86, wherein the first wash buffer applied to the chromatography support has a pH of 7.2 and comprises about 20 mM Tris-HCl and about 250 mM NaCl.

141. The method of any one of claims 78-86, wherein the second wash buffer applied to the chromatography support has a pH of 5.2 and comprises about 50 mM glycine, about 100 mM acetic acid, and about 175 mM NaCl.

142. A composition comprising the IαIps produced by the method of any one of claims 1-141.

143. The composition of claim 142, wherein the composition is suitable for administration to a human.

144. A pharmaceutical composition comprising the composition of claim 142 or 143 and a pharmaceutically acceptable excipient.

145. A method of treating a disease or condition in a subject in need thereof comprising administering to the subject the composition of claim 142 or 143 or the pharmaceutical composition of claim 144.

146. A kit comprising the composition of claim 142 or 143 or the pharmaceutical composition of claim 144.

147. The kit of claim 146, wherein the kit further comprises instructions for therapeutic use.

148. A method of purifying an IαIp from plasma comprising:
(a) diluting the plasma with a dilution buffer comprising deionized water to form diluted plasma;
(b) applying the diluted plasma to an ETOXICLEAR™ resin and separating a flow through comprising the diluted plasma that does not bind to the ETOXICLEAR™ resin;
(c) applying a first wash buffer comprising about 75 mM glycine, about 100 mM AcOH, and about 150 mM NaCl at a pH of about 5.2 to the ETOXICLEAR™ resin and separating a flow through comprising the first wash buffer;
(d) applying a second wash buffer comprising about 20 mM Tris-HCl and about 300 mM NaCl at a pH of about 7.2 to the ETOXICLEAR™ resin and separating a flow through comprising the second wash buffer; and
(e) applying an elution buffer comprising about 20 mM Tris-HCl and about 500 mM NaCl at a pH of 7.2 to the ETOXICLEAR™ resin and collecting an eluate comprising the IαIp.

149. A method of purifying an IαIp from plasma comprising:
(a) diluting the plasma with a dilution buffer comprising 15 mM phosphate at a pH of about 5.5 to the plasma to form diluted plasma;
(b) applying the diluted plasma to a DETOXI-GEL™ resin and separating a flow through comprising the diluted plasma that does not bind to the DETOXI-GEL™ resin;
(c) applying a first wash buffer comprising about 15 mM phosphate and about 50 mM NaCl at a pH of about 5.5 to the DETOXI-GEL™ resin and separating a flow through comprising the first wash buffer;
(d) applying a second wash buffer comprising about 15 mM phosphate and about 100 mM NaCl at a pH of about 5.5 to the DETOXI-GEL™ resin and separating a flow through comprising the second wash buffer; and
(e) applying an elution buffer comprising about 15 mM phosphate and about 1,000 mM NaCl at a pH of about 5.5 to the DETOXI-GEL™ resin and collecting an eluate comprising the IαIp.
